(19) United States
(12) Patent Application Publication
(10) Pub. No.: US 2002/0034725 A1
(43) Pub. Date: Mar. 21, 2002

(54) SENSITIZATION OF CELLS TO RADIATION AND AND CHEMOTHERAPY

(76) Inventors: W. GILLIES MCKENNA, PHILADELPHIA, PA (US); RUTH J. MUSCHEL, PHILADELPHIA, PA (US); ERIC J. BERNHARD, PHILADELPHIA, PA (US); SAID M. SEBTI, TAMPA, FL (US); ANDREW D. HAMILTON, PITTSBURGH, PA (US)

Correspondence Address: AKIN, GUMP, STRAUSS, HAUER & FELD, L.L.P., ONE COMMERCE SQUARE, 2005 MARKET STREET, SUITE 2200, PHILADELPHIA, PA, 19103, US

(21) Appl. No.: 08/839,248

(22) Filed: Apr. 15, 1997

Related U.S. Application Data
(60) Provisional application No. 60/015,477, filed on Apr. 15, 1996.

(51) Int. Cl. A61M 36/14; A61K 51/00; A01N 37/18; A61K 38/00; C12Q 1/00; C12Q 1/68; G01N 33/53; A61K 38/16; A61K 31/70; A01N 43/04; A61K 39/395; A61K 31/545; G01N 33/52

(52) U.S. Cl. 435/4; 514/2; 514/44; 435/6; 435/7.1; 424/130.1; 424/1.11

(57) ABSTRACT

The invention relates to a method of conferring radiation sensitivity on a tumor cell comprising administering to the cell an inhibitor of a protein product which participates in the ras signalling pathway, whereby inhibition of the protein product confers radiation sensitivity on the cell.

### CROSS REFERENCE TO RELATED APPLICATIONS

This application claims priority under 35 U.S.C. § 119(e) to U.S. Provisional Application No. 60/015,477, filed on Apr. 15, 1996.

### GOVERNMENT SUPPORT

The invention was supported in part by funds from the U.S. Government (National Institutes of Health Grant No. RO1 CA 64227) and the U.S. Government may therefore have certain rights in the invention.

### FIELD OF THE INVENTION

The field of the invention is radiation and chemotherapy.

### BACKGROUND OF THE INVENTION

Radiation therapy and chemotherapy are effective tools for the treatment of many types of cancers, but the success of this type of treatment in ablating tumor growth is limited by the intrinsic resistance of cells to either or both procedures.

Radiation resistance in cells may be the result of the presence in cells of activated oncogenes; however, this factor alone does not account for the increased radiation resistance in all tumor cells. For example, in tissue culture, the expression of ras oncogenes has been shown to increase radioresistance in NIH 3T3 cells (Fitzgerald et al., 1985, Am. J. Clin. Oncol. 8:517-522; Sklar et al., 1988, Science 239:645-647; Pirollo et al., 1993, Radiat. Res. 135:234-243; Samid et al., 1991, Radiat. Res. 126 244-250), rat embryo fibroblasts (McKenna et al., 1990, Int. J. Rad. Onc. Biol. Phys. 18:849-860; Ling et al., 1989, Radiat. Res. 120:267-279), rhabdomyosarcoma cells (Hermens et al., 1992, Cancer Res. 52:3073-3082), human osteosarcoma cells (Miller et al., 1993, Int. J. Cancer 53:302-307; Miller et al., 1993, Int. J. Radiat. Biol. 64:547-554) and mammary carcinoma cells (Bruyneel et al., 1993, Eur. J. Cancer 29A: 1958-1963). In contrast, the presence of K-ras in rat kidney epithelial cells rendered these cells more sensitive to radiation (Harris et al., 1990, Somat. Cell & Molec. Genet. 16:39-48). Further, human mammary epithelial cells also exhibited no increase in radioresistance in the presence of ras (Alapetite et al., 1991, Int J. Radiat. Biol. 59:385-396).

In human fibroblast cell lines, unlike rodent cell lines, transfection with H-ras alone resulted in neither transformation nor radioresistance of the cells (Su et al., 1992, Int. J. Radiat Biol. 62:201-210). The human HaCaT keratinocyte line transfected with H-ras also exhibited little change in radiosensitivity when treated with high doses of radiation. However, low dose irradiation of these cells resulted in a modest increase in radiation resistance (Mendonca et al., 1991, Int. J. Radiat Biol. 59:1195-1206).

The presence in cells of oncogenes other than ras which are involved in the ras signaling pathway, may also be associated with resistance of cells to radiation. Such oncogenes include raf (Kasid et al., 1989, Science 243:1354-1356; Pirollo et al., 1989, International Journal of Radiation Biology 55:783-796), mos Pirollo et al., 1989, supra; Suzuki et al., 1992, Radiation Research 129:157-162), ets, and sis (Pirollo et al., 1993, supra).

Some ras mutations may result in cell transformation and other ras mutations may not result in cell transformation. Mutations in ras which result in the formation of tumors are those which give rise to an activated form of ras protein, which protein promotes transformation of the ras-expressing cell and therefore, the formation of tumors derived therefrom.

Mutations in H- and K-ras are frequently found in human tumors of both epithelial and mesenchymal origin (Bos, 1989, Cancer Res. 49:4682-4689). H-ras mutations have been detected in as many as 45% of bladder cancers with the greatest occurrence in higher grade malignancies (Czerniak et al., 1992, Human Pathol. 23:1199-1204). H-ras mutations are also seen in thyroid (Lemoine et al., 1989, Oncogene 4:159-164), head and neck cancers (Anderson et al., 1992, J. Otolaryngol. 21:321-326), and sarcomas (Wilke et al., 1993, Modem Pathol. 6:129-132; Bohle et al., 1996, Am. J. Pathol. 148:731-738, 1996), prostate (Konishi et al., 1995, Am. J. Pathol. 147:1112-1122; Watanabe et al., 1994, Int. J. Cancer 58:174-178) and cervical (Riou et al., 1988, Oncogene 3:329-333) carcinomas. Mutations in K-ras have an even higher prevalence in human tumors, occurring in 75-95% of pancreatic cancers (Smit et al., 1988, Nucleic Acids Res. 16:7773-7782; Capella et al., 1991, Environ. Health Perspec. 93:125-131) and 50% of colorectal tumors (Capella et al., 1991, supra; Vogelstein et al., 1988, New Engl. J. Med. 319:525-532). A significant incidence of K-ras mutations has also been reported in adenocarcinoma of the lung (Husgafvel-Pursiainen et al., 1995, J. Occup. Environ. Med. 37:69-76), later stage cervical tumors (III and IV) (Symonds et al., 1992, Eur. J. Cancer 28A:1615-1617; Hiwasa et al., 1992, Eur. J. Gynaec. Oncol. 13:241-245) and prostate tumors (Konishi et al., 1995, supra; Watanabe et al., 1994, supra).

It is known in the art that many proteins require post-translational modification for their activity. Such post-translational modification may include farnesylation and geranylgeranylation of a protein. The enzyme, farnesyl transferase (FiTase), is responsible for farnesylation of proteins (Reiss et al., 1990, Cell 62:81-88; Reiss et al., 1991, Proc. Natl. Acad. Sci. USA 88:732-736; Moores et al., 1991, J. Biol. Chem. 266:14603-14610) and the enzyme geranylgeranyl transferase (GGTase) is responsible for the geranylgeranylation of proteins (Moomaw et al., 1992, J. Biol. Chem. 267:17438-17443; Yokoyama et al., 1991, Proc. Natl. Acad. Sci. USA 88:5302-5306; Yokoyama et al., 1993, J. Biol. Chem. 268:4055-4060).

The protein substrates of FTase all share the common feature of having a CAAX sequence at the carboxyl terminal where X is most often a methionine, serine, cysteine, alanine or glutamine (Reiss et al., 1990, supra; Reiss et al., 1991, supra; Moores et al., 1991, supra). Proteins which terminate in CAAX sequences wherein X is leucine or isoleucine may be modified by the addition thereto of the 20 carbon cholesterol biosynthesis intermediate geranylgeranyl pyrophosphate, which is added to the protein via GGTase (Moomaw et al., 1992, supra; Yokoyama et al., 1991, supra; Yokoyama et al., 1993, supra).

Each of the known mammalian ras genes, H, N, K_{A} and K_{B} (Barbacid, 1987, Ann. Rev. Biochem. 56:779-827) contain at the carboxyl terminus a posttranslational signal in the form of a CAAX box where C is cysteine, A is valine, leucine or isoleucine and X is methionine or serine (Hancock et al., 1989, Cell 57:1167-1177; Hancock et al., 1990, Cell 63:133-139). Although H-ras is exclusively farnesylated, at least one other ras protein, K_{B}-ras, can be both farnesylated and geranylgeranylated (James et al., 1995, J. Biol. Chem. 270:6221-6226; Lerner et al., 1995, J. Biol. Chem. 270:26770-26773; Lerner et al., J. Biol. Chem. 270:26802-26806).

Posttranslational modification of ras may be inhibited using inhibitors of either farnesylation or geranylgeranylation of ras (Gibbs et al., 1994, Cell 77:175-178; Buss et al., 1995, Chem. Biol. 2:787-791; Hamilton et al., 1995, Drug News and Perspectives 8:138-145). In particular, peptidometics that disrupt protein-protein interaction, most particularly, those which mimic the CAAX structure at the carboxyl terminus of oncogenes, have been the subject of intense investigation (Reiss et al., 1990, supra). These peptides are known to, or are predicted to inhibit posttranslational modification of some oncogenes.

The most common treatments for cancer in animals, including humans are surgical excision of the tumor, irradiation of the tumor and the administration of chemotherapy. There is an acute need to provide improvements to these approaches, in particular, there is an acute need to improve the effectiveness of radiation and chemotherapy in cancer patients. The present invention satisfies this need.

### SUMMARY OF THE INVENTION

The invention relates to a method of conferring radiation sensitivity on a tumor cell comprising administering to the cell at least one inhibitor of a protein product which participates in the ras signalling pathway, whereby inhibition of the protein product confers radiation sensitivity on the cell.

The invention also relates to a method of reducing the growth of a tumor in an animal comprising administering to the animal at least one inhibitor of a protein product expressed in cells of the tumor, which protein product participates in the ras signalling pathway, and whereby inhibition of the protein product confers radiation sensitivity on the cells, wherein the inhibitor is administered to the animal in an amount sufficient to effect inhibition of the protein product, and the animal is irradiated thereby reducing the growth of the tumor in the animal.

Also included in the invention is a method of eliminating a tumor from an animal comprising administering to the animal at least one inhibitor of a protein product expressed in cells of the tumor, which protein product participates in the ras signalling pathway and whereby inhibition of the protein product confers radiation sensitivity on the cells, wherein the inhibitor is administered to the animal in an amount sufficient to effect inhibition of the protein product, and the animal is irradiated thereby eliminating the tumor from the animal.

The invention further relates to a method of identifying a prenylation inhibitor which confers radiation sensitivity on a cell population comprising providing a population of cells which express a protein in need of prenylation for activity of the protein and which protein participates in the ras signalling pathway, adding to the cells a test compound, irradiating the cells, and measuring the level of sensitivity of the cells to irradiation, wherein a higher level of radiation sensitivity in cells administered the test compound compared with the level of radiation sensitivity in cells which were not administered the test compound, is an indication that the test compound confers radiation sensitivity on the cell population.

In one aspect of the invention, the animal is a human.

In another aspect of the invention, the protein product is an oncogene protein product.

In one embodiment, the oncogene protein product is a ras protein, which may be selected from the group consisting of H-ras, K_{A}-ras, K_{B}-ras and N-ras.

In yet another embodiment, the protein product is selected from the group consisting of rhoA, rhoB, rhoC and RAC-1.

In another aspect of the invention, the inhibitor is an antisense oligonucleotide or the inhibitor is a ribozyme.

In another embodiment of the invention, the protein product has at the carboxyl terminus of the protein the sequence CAAX, wherein C is cysteine, A is an aliphatic amino acid, valine, leucine or isoleucine and X is methionine, serine, cysteine, alanine, glutamine, leucine or isoleucine.

In yet another embodiment of the invention, the inhibitor is a protein prenylation inhibitor which may be a farnesylation inhibitor, which is preferably selected from the group consisting of FTI-276 and FTI-277. In addition, the farnesylation inhibitor may comprise FTI-276 and FTI-277 having any sulfate groups thereon removed.

The prenylation inhibitor may also be a geranylgeranylation inhibitor, which is preferably selected from the group consisting of GGTI-297 and GGTI-298. In addition, the geranylgeranylation inhibitor may comprise GGTI-297 and GGTI-298 having any sulfate groups thereon removed.

In yet another aspect of the invention, the tumor is a solid tumor which may be selected from the group consisting of prostate, lung, colon, breast, pancreas, cervical carcinoma, cervical sarcoma, rectum, colon, ovary, bladder, thyroid, head and neck. Preferably, the tumor is selected from the group consisting of lung, pancreas, colon and rectum.

### BRIEF DESCRIPTION OF THE DRAWINGS

FIG. 1A is a drawing depicting the chemical structures of the peptidometic farnesyl transferase inhibitors L-731,735, B581 and L-739,750.

FIG. 1B is a drawing depicting the chemical structure of the peptidometic farnesyl inhibitor L-744,832.

FIG. 2 is a drawing depicting the chemical structures of the peptidometic farnesyl transferase inhibitors FTI-205, FTI-249, FTI-254, FTI-276, FTI-277, B956, B1086, BZA-2B, and BZA-5B.

FIG. 3 is a drawing depicting the chemical structures of the peptidometic farnesyl transferase inhibitors FTI-265, FTI-281, FTI-289, and L745,631

FIG. 4 is a drawing depicting the chemical structures of the peptidometic farnesyl transferase inhibitors BMS-185878, BMS -184467, and BMS-193269.

FIG. 5 is a drawing depicting the chemical structures of twofarnesyl pyrophosphate analogs, the farnesyl transferase inhibitors, 2-hydroxyfarnesylphosphonic acid and farnesylmethylhydroxyphosphinyl methyl phosphonic acid.

FIG. 6 is a drawing depicting the chemical structures of four farnesyl transferase inhibitors which were obtained from natural product or chemical library screens. These inhibitors include chaetomellic acid A, Zaragozic acid A analog, Manumycin, and SCH-44342.

FIG. 7 is a drawing depicting the chemical structures of the peptidometic geranylgeranyl transferase I inhibitors GGTI-279, GGTI-280, GGTI-287, GGTI-286, GGTI-297, and GGTI-298.

FIG. 8A is an image of a Western blot depicting a time course of the shift in mobility of the ras protein from the farnesylated form to the unfarnesylated form. 5R cells transformed with H-ras^{v12} oncogene were treated with 5 μM FTI-277. At the times indicated (hours), samples were harvested and cell lysates were prepared for Western blot analysis using anti-H-ras antibody for detection of H-ras. The upper band in the gel corresponds to unfarnesylated H-ras protein. C indicates control cells.

FIG. 8B is an image of a Western blot depicting a time course of the shift in mobility of the ras protein from the unfarnesylated form to the farnesylated form following removal of the farnesylation inhibitor from the cell culture. 3.7 cells co-transformed with H-ras^{v12} plus v-myc oncogenes were treated with 5 μM FTI-277 for 30 hours prior to removal of the inhibitor from the medium. At the times indicated after removal (hours), samples were harvested and cell lysates were prepared for Western blot analysis using anti-H-ras antibody for detection of H-ras.

FIG. 8C is an image of a Western blot depicting the effects on of the inhibitor, FTI-277, on ras farnesylation in 3.7, 4R, 5R, MR4, REF, and REF-GG cells following 24 hours of treatment with FTI-277 at the indicated concentrations. Cells in log phase culture were treated with the indicated dose of FTI-277 (μM). After 24 hours, samples were harvested and cell lysates were prepared for Western blot analysis. H-ras specific antibody was used to detect ras in all cell types except for MR4 cells where pan ras specific antibody results are shown due to very low levels of H-ras expression in these cells.

FIG. 9 is an image of a Western blot depicting the effects of the inhibitor, L-744,832, on ras farnesylation in 3.7, 5R, and MR4 cells following 24 hours of treatment with the inhibitor. Cells in log phase culture were treated with the indicated dose of inhibitor (μM). After 24 hours, samples were harvested and cell lysates were analyzed by Western blotting using an H-ras specific monoclonal antibody. The MR4 cell blot was exposed 20 times longer than 3.7 or 5R cell blots.

FIG. 10 is an image of a Western blot depicting the effects of the inhibitor, GGTI-286, on ras farnesylation in 5R and REF-GG cells following treatment with the indicated concentrations (μM) of the inhibitor for 24 hours. Following the 24 hour treatment, samples were harvested and cell lysates were analyzed by Western blotting using a H-ras specific monoclonal antibody. U-F: Unfarnesylated H-ras; U-GG: Ungeranylgeranylated ras-GG. The chimeric H-ras^{v12} migrated slightly faster than the farnesylated H-ras in the gel shown; thus, the unprenylated H-ras from REF-GG co-migrated with the farnesylated H-ras from 5R cells.

FIG. 11A is an image of 3.7 cells that were cultured in medium containing 2.5 μM FTI-277 (right) or DMSO (left) for 48 hours.

FIG. 11B is an image of REF-GG cells that were cultured in medium containing 5 μM FTI-277 (right) or DMSO (left) for 48 hours.

FIG. 12A is a graph depicting the effects on apoptosis of REF and 3.7 cells following treatment with the indicated concentrations of FTI-277 and irradiation of the cells with 10REF-GG. Apoptosis was quantitated 24 hours after treatment by scoring for changes in nuclear morphology following staining of the cells with propidium iodide.

FIG. 12B is a graph depicting the effects on apoptosis of 4R, 5R, and REF-GG cells following treatment with the indicated concentrations of FTI-277 and irradiation of the cells with 10 Gray. Apoptosis was quantitated 24 hours after treatment by scoring for changes in nuclear morphology following staining of the cells with propidium iodide.

FIG. 13A is an image of a Western blot depicting the effects of FTI-277 on ras farnesylation in cultured cells derived from a primary and a metastatic tumor following 24 hours of treatment with the inhibitor at the indicated concentrations. Mouse prostate tumor cells transformed with H-ras^{v12} and myc oncogenes were treated with the indicated doses (μM) of FTI-277. After 24 hours, samples were harvested for Western blot analysis using an anti-H-ras antibody. The upper band (arrow) corresponds to unfarnesylated H-ras. C denotes controls.

FIG. 13B is a graph depicting the effects of the inhibitor, FTI-277, at the concentrations indicated (μM) on radiation induced apoptosis of prostate tumor cells. Cells were treated with FTI-277 for 24 hours before irradiation with 10 Gray. Apoptosis was quantitated 24 hours after irradiation by scoring for changes in nuclear morphology following staining of the cells with propidium iodide. Panel A depicts prostate tumor cells cultured from a primary tumor. Panel B depicts prostate tumor cells cultured from an isolated metastasis derived from the tumor.

FIG. 14A comprises a series of graphs depicting clonogenic survival of 5R, 3.7, MR4, and REF cells following treatment with FTI-277 and irradiation of the cells. Immediately prior to irradiation, FTI-277 was added at concentrations of 2.5 μM (3.7 cells) or 5 μM (5R, MR4 and REF cells). The inhibitor was diluted out of the culture medium 24 hours later resulting in a final concentration of inhibitor of 1 μM (3.7 cells) or 2 μM (5R, MR4 and REF). The plating efficiencies of MR4 and SR cells were unaffected by treatment with FTI-277, being 100% and 32-38%, respectively. FTI-277 reduced the plating efficiency of 3.7 and REF cells by 50% of untreated control values which were 75% and 5%, respectively. These results are not due to any toxic effects of the drug. The data points shown represent the mean of the results obtained from at least three separate dishes of cells. The open symbols indicate the results obtained in untreated cells and the closed symbols are those results obtained in cells treated with FTI-277. In the panel labeled 3.7, the open triangles are the results obtained in untreated MR4 cells.

FIG. 14B comprises two graphs depicting clonogenic survival of REF-GG cells following treatment with GGTI-298 and irradiation. REF-GG cells were plated at 1 to 5 cells per well in microtiter plates in the absence (top panel) or presence (bottom panel) of 8 μM of the geranylgeranyltransferase inhibitor, GGTI-298. Cells were then irradiated with 2 Gray or were mock irradiated. Cells were re-fed after 24 hours with medium that contained no inhibitor, thus diluting the inhibitor to 0.8 μM. These cells were then incubated for two weeks prior to scoring for colony formation. The data are presented as the natural log of the fraction of negative wells. The surviving fraction at 2 Gray was determined from the differences in the slopes obtained by linear regression of analysis of irradiated and unirradiated cells. The surviving fraction at 2 Gray calculated for cells irradiated after GGTI-298 treatment was 0.64. Control cell surviving fraction after 2 Gray was 0.91. The correlation coefficient for linear regression analysis (r²) was greater than 0.95 in all cases.

FIG. 14C is a series of graphs depicting the effect of 5 μM FTI-277 and 2 Gray irradiation on murine prostate tumor cells cultured from a metastic lung nodule. Cells were referred at 24 hours following irradiation in order to dilute the concentration of the inhibitor to 0.5 μM. The surviving cell fraction was assessed as described in FIG. 14B. The correlation coefficient for linear regression analysis (r²) was greater than 0.95 in all cases.

FIG. 15A is a graph depicting growth of primary REF cells (circles) and 3.7 cells (squares) after FTI-277 treatment. Two×10⁵ cells were plated in medium containing 2.5 μM (3.7 cells) or 5 μM (REF cells) of FTI-277. Medium was diluted after 24 hours to such that the concentration of inhibitor was significantly reduced. Cells were harvested from replicate dishes at one day intervals and counted using a hemocytometer to determine the total cell number in each culture. Open symbols: DMSO (drug carrier) treated cells; closed symbols: FTI-277 treated cells.

FIG. 15B is a graph depicting growth of MR4 cells (circles) and 5R cells (squares) following treatment with FTI-277 treatment. Three×10⁵ cells were plated in medium containing 5 μM FTI-277. Cells were harvested from replicate dishes at one day intervals and counted using a hemocytometer to determine the total cell number in each culture. Open symbols: DMSO (drug carrier) treated cells; closed symbols: 5 μM FTI-277 treated cells.

FIG. 16A is an image of a Western blot depicting changes in farnesylation of H-ras^{v12} following treatment of human bladder carcinoma cells with FTI-277. T24 bladder carcinoma cells were treated with 5 μM of FTI-277 for the times indicated (hours). Samples were harvested and cell lysates were prepared for Western blot analysis using anti-H-ras antibody. Untreated control samples harvested at 0 and 30 hours are shown for comparison.

FIG. 16B is a graph depicting colony formation following FTI-277 treatment and irradiation of human T24 bladder carcinoma cells. Cells were treated for 24 hours with 5 μM of FTI-277 and were harvested and plated at the indicated cell density in medium containing DMSO (left panel) or 5 μM FTI-277 (right panel) and were immediately irradiated. Cultures were re-fed after 24 hours with medium that contained no inhibitor. This resulted in a final inhibitor concentration of 0.5 μM in the medium. The cells were allowed to grow for two weeks prior to scoring for colony formation. Open squares: unirradiated cells; closed circles: 2 Gray irradiated cells. The surviving fraction of cells was calculated as described in the description of FIG. 14B. The correlation coefficient for linear regression analysis (r²) was greater than 0.95 in all cases.

FIG. 17A is an image of a Western blot depicting inhibition by FTI-277 of K-ras prenylation in human SW480 colon carcinoma cells. SW480 cells were treated with the indicated concentrations of FTI-277 (μM) for 48 hours. Samples were harvested and cell lysates were analyzed by Western blotting using either a H-ras monoclonal antibody (top) or a K-ras monoclonal antibody (bottom). Arrows indicate unfarnesylated ras bands.

FIG. 17B is a graph depicting a reduction in radiation survival of SW480 colon carcinoma cells following FTI-277 inhibition of K-ras prenylation. SW480 cells were treated for 24 hours with 30 μM FTI-277 before irradiation. Clonogenic survival was subsequently assessed in the cells. Treatment with the inhibitor was maintained for 24 hours after irradiation, at which time medium was replaced with inhibitor free medium. Control cells were treated as above with diluent. Open squares: control cells; closed squares: FTI-277 treated cells.

FIG. 18A is an image of a Western blot depicting the specific inhibition of K-ras prenylation by combined FTI-277 and GGTI-298 treatment. Log phase cultures of human pancreatic carcinoma cells (Panc-1) and colon carcinoma cells (SW480) were treated with 5 μM FTI and 8 μM GGTI 298 for 48 hours. Cell samples were then harvested and cell lysates were prepared for Western blot analysis using monoclonal antibodies to K-ras and nuclear lamin B. The electrophoretic mobility of the K-ras mutant in SW480 is slower than that of the mutant K-ras in Panc-1 cells.

FIG. 18B is a graph depicting a reduction in the radiation survival of SW480 cells following inhibition of K-ras prenylation by FTI-277 and GGTI-298. SW480 cells were treated for 24 hours with 5 μM FTI-277 and 8 μM GGTI-298 before irradiation and assessment of clonogenic survival. Inhibitor treatment was maintained for 24 hours after irradiation, at which time the medium was replaced with inhibitor free medium. Control cells were treated as above with an equal amount of drug-free diluent. Open squares: control cells; closed squares: FTI and GGTI treated cells.

FIG. 18C is a graph depicting colony formation in A549 human lung cancer cells treated with FTI-277 and GGTI-298. Cells were plated at the indicated cell numbers per well in 96 well microtiter plates in the presence (panel B) or absence (panel A) of 5 μM FTI-277 and 8 μM GGTI-298. Cells were then irradiated with 2 Gray (closed symbols) or mock irradiated (open symbols). Twenty-four hours after irradiation, cultures were fed with medium without inhibitor resulting in a 10-fold dilution of inhibitor in the culture. Colonies of cells were scored after three weeks of growth. The surviving fraction of cells was calculated as described in the description of FIG. 14B. The correlation coefficient for linear regression analysis (r²) was greater than 0.97 in all cases.

FIG. 19A is an image of a Western blot depicting the detection of H-ras in transformed rat embryo fibroblast tumor tissue grown in nude mice. Ras expression was analyzed by Western blotting using anti-H-ras monoclonal antibody in lysates obtained from various cells as follows: Lane 1: 5R cells grown in tissue culture. Lane 2: 5R cells grown as tumors in nude mice. Lane 3: normal mouse liver tissue which serves as a negative control. Fifty μg protein was loaded in each lane. The migration of molecular weight standards is indicated on the left (kDa.).

FIG. 19B is an image of a Western blot depicting altered H-ras migration in human tumors grown in nude mice. Expression of H-ras was detected by Western blot analysis using anti-H-ras monoclonal antibody of lysates of the human colon adenocarcinomas, SW480 and LoVo, grown in nude mice. Lysates were obtained from tumors excised from a vehicle (DMSO) treated mouse (Lane 1) and mice treated twice with intraperitoneal injections of 50 mg/kg of FTI-277 18 hours after treatment was initiated (Lanes 2 and 3). Lane 1 and 2: SW480, Lane 3: LoVo. The arrow denotes the migration of unfarnesylated H-ras.

### DETAILED DESCRIPTION OF THE INVENTION

It has been discovered in the present invention that compounds which inhibit the function of selected oncogenes render cells more susceptible to radiation and chemotherapy. In particular, it has been discovered that compounds which inhibit posttranslational modification of selected oncogenes render cells more susceptible to radiation and chemotherapy.

Thus, the present invention provides a method of killing tumor cells, wherein cells are administered an inhibitor of an oncogene in combination with conventional radiation or chemotherapy. While inhibitors of oncogene posttranslation are candidate anti-tumor agents and conventional radiation or chemotherapy are known anti-cancer treatments, it has been discovered in the present invention that the administration of an inhibitor of an oncogene to a tumor cell in combination with radiation therapy is superior in effecting death of the cell when compared with treatment of the cell with radiation alone. For reasons which are presented herein, the present invention is also applicable to chemotherapy killing of tumor cells.

The method of the invention is thus useful for effecting reducing tumor growth or eliminating (i.e., ablating) a tumor in an animal. Further, in order to reduce tumor growth or eliminate a tumor in an animal, less radiation and/or chemotherapy may be required to treat the animal than has heretofore been possible, thereby reducing the level of deleterious side effects experienced by the animal undergoing treatment.

By the term “reduction of tumor growth” or “reducing tumor growth” as used herein, means a reduction in the rate of growth of a tumor or a reduction in the overall size of a tumor when the tumor has been administered the inhibitor of the invention combined with radiation or chemotherapy, when the rate of growth of or the size of the tumor is compared with the rate of growth of or the size of a tumor which has not been administered the inhibitor.

By the term “elimination of a tumor” or “ablation of a tumor” as used herein, means that the presence of the tumor in animal cannot be be detected using ordinary tumor detection technology known in the art at the time of the present invention.

The method of the invention thus provides a heretofore unknown means of acute cancer therapy, wherein target tumor cells in the animal are sensitized by the administration of the inhibitor and are subsequently killed by either radiation or chemotherapy.

The inhibitors which are useful in the present invention are those which inhibit the function of an oncogene protein in a cell, which ongogene protein is responsible for the radiation and/or chemotherapy resistance of the cell.

The types of inhibitors include those which inhibit production of the oncogene protein, including, but not limited to, antisense oligonucleotides which specific for the subject oncogene mRNA. Anti-oncogene ribozymes are also included in the invention as inhibitors of oncogene protein production.

The preferred oncogene protein inhibitors of the invention are those inhibitors which inhibit posttranslational modification including prenylation (farnesylation or geranylgeranylation) of the oncogene protein. However, in addition to prenylation of ras as a target for inhibition of ras activity (members of the ras family being the preferred oncogenes), palmitoylation, which occurs subsequent to farnesylation of ras, may also be used as a target for inhibition of ras activity (Gelb, 1997, Science 275:1750-1751).

Inhibition of protein function using an antisense approach is well known and is accepted in the art of modulation of protein function. Antisense oligonucleotides are known to enter cells and to be effective in regulating expression of a target gene against which they are directed (Wagner, 1994, Nature 372:333-335). In fact, in at least one instance, administration of an antisense oligonucleotide to a human has resulted in demonstrated efficacy against cytomegalovirus-associated retinitis (Antiviral Agents Bulletin 5: 161-163, 1992; BioWorld Today, Dec. 20, 1993). Thus, pharmaceutical compositions comprising antisense oligonucleotides are considered by those in the art to be both safe and efficacious in humans (Cohen et al., December 1994, Scientific American, pp. 76).

Antisense inhibitors of ras function preferably include oligonucleotides which are directed against the 5′ portion of the mRNA specifying the specific ras protein against which the inhibitor is directed. Since the nucleotide sequence of the ras oncogenes is known, the development of antisense oligonucleotides having specificity for the 5′ portion of ras mRNA is well within the skill of those in the art of antisense technology. The anti-ras oligonculeotide may also be modified to enhance its stability and to enhance the efficiency with which it enters cells, etc., also using protocols which are available to those in the art of antisense technology.

Similarly, ribozymes directed against ras are known in the art and are therefore useful as inhibitors to confer radiation and/or chemottherapy sensitivity on tumor cells (Barinaga, 1993, Science 262:1512-1514; Pyle, 1993, Science 261:709-714; Kijima et al., 1995, Pharmac. Ther. 68:247-267).

The term “inhibition of a protein product” as used herein, means inhibition of the activity of a subject protein. For example, when the protein is an enzyme, inhibition of the activity of the protein means inhibition of enzyme activity. The term should not be construed to mean complete inhibition of the activity of the protein product. Rather, the term should be construed to mean that the level of activity of the protein product is reduced either partially or completely in the presence of the inhibitor of the protein product, compared with the level of activity of the protein product in the absence of the inhibitor.

By the term “in an amount sufficient to effect inhibition of the protein product” as used herein as it refers to an inhibitor, is meant a concentration of inhibitor which inhibits the activity of the protein product as defined herein.

Preferably, the posttranslational modification inhibitors which are useful in the methods of the invention are those which inhibit farnesylation or geranylgeranylation of the oncogene protein. Thus, the method of the invention more particularly includes inhibitors of FTase or GGTase, or inhibitors of both enzymes. Farnesylation and geranylgeranylation of proteins is collectively known as prenylation. FTase and GGTase are the enzymes which catalyze prenylation of oncogene protein products thus, the inhibitors which are most useful in the methods of the invention are referred to herein as “prenylation inhibitors.”

While any inhibitor of a subject oncogene protein product may be useful for sensitization of tumor cells to radiation and/or chemotherapy, the discussion which follows uses as an example, oncogene protein prenylation inhibitors, it being understood that the methods of the invention should not be construed as being limited solely to these types of inhibitors.

According to the present invention, inhibition of prenylation of an oncogene protein product sensitizes cells to radiation thereby enhancing the effectiveness of the radiation in effecting death of the cell. The mechanism by which inhibition of prenylation of an oncogene protein sensitizes cells to radiation is unknown. While not wishing to be bound by any theory, it is thought that the enhanced radiation sensitivity of cells in which posttranslational modification of an oncogene protein such as ras is inhibited, is the result of an affect of the inhibitor on the cell cycle. For example, in the case of the ras or the myc protein, when either of these proteins is activated in a cell, the cell remains in the G2 phase of the cell cycle for a longer time compared with the time spent in G2 by a cell in which either of these proteins is not activated (McKenna et al., 1991, Radiat. Res. 125: 283-287). Cells which remain in G2 do not replicate DNA; therefore, these cells are more resistant to radiation therapy because radiation therapy relies for its effect, on ongoing DNA replication in the cell. Inhibition of ras or myc promotes egress of the cell from the G2 phase of the cell cycle, thereby facilitating DNA replication in the cell which subsequently confers radiation sensitivity on the cell.

The methods of the invention should therefore be construed to include the use of any and all protein prenylation inhibitors which inhibit activation of an oncogene in a cell, which oncogene when activated, causes the cell to remain in the G2 phase of the cell cycle for a longer period of time than that in a cell in which the oncogene is not activated.

Given that an inhibitor of prenylation of an oncogene in a cell causes the cell to exit the G2 phase of the cell cycle and eventually resume DNA replication, the methods of the invention should also be construed to include the use of chemotherapy as a means of enhancing tumor cell death when the chemotherapy relies for its effect on DNA replication of the cell.

The terms “prenylation inhibitor” or “inhibitor of prenylation” as used here, mean a compound which inhibits the attachment of an isoprenoid moiety to a protein.

An “isoprenoid moiety” as used herein, should be construed to mean a farnesyl or a geranylgeranyl moiety.

The term “farnesylation inhibitor” as used herein, means a compound which inhibits the attachment of a farnesyl moiety to a protein.

The term “geranylgeranylation inhibitor” as used herein, means a compound which inhibits the attachment of a geranylgeranyl moiety to a protein.

As discussed herein, inhibitors of protein prenylation which are useful in the present invention include inhibitors of FTase and GGTase, both of which function to transfer farnesyl or geranylgeranyl moieties to the amino acid sequence CAAX at the carboxyl terminus of an oncogene protein, wherein C is cysteine, A is an aliphatic amino acid, valine, leucine or isoleucine and X is methionine, serine, cysteine, alanine or glutamine when CAAX is a FTase substrate and X is leucine or isoleucine, when CAAX is a GGTase substrate.

Thus, one embodiment of the method of the invention includes the use 25 of peptidometics comprising the tetrapeptide, CAAX, or analogs thereof. It is known that CAAX may be farnesylated by FTase as efficiently as the corresponding full length protein. Moreover, it is known that CAAX is a potent competitive inhibitor of FTase (Reiss et al., 1990, supra).

Modifications of the CAAX peptide are useful in the methods of the present invention provided such modifications give rise to a peptide which inhibits prenylation of an oncogene protein product in the prenylation assays described in the experimental examples provided herein. For example, conservative amino acid changes may be made in the peptide, which although they alter the primary sequence of the protein or peptide, do not normally alter its function. Conservative amino acid substitutions typically include substitutions within the following groups:

glycine, alanine;

valine, isoleucine, leucine;

aspartic acid, glutamic acid;

asparagine, glutamine;

serine, threonine;

lysine, arginine;

phenylalanine, tyrosine.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

In addition to the tetrapeptide CAAX described herein, peptides having different amino acid lengths are also included in the invention provided they inhibit prenylation of an oncogene protein product in a cell as assessed in the prenylation assays described in the experimental details presented herein. Such peptides may comprise at least two amino acids in length, which amino acids are derived from the carboxyl terminus of a subject oncogene protein product whose prenylation is to be inhibited. Such peptides may comprise an amino acid length which is greater than two amino acids, i.e., which is between two amino acids and fifteen amino acids in length. The peptides may comprise between three and eleven amino acids in length, between four and ten amino acids in length, or between about five and nine amino acids in length. Preferably, the peptide is about four amino acids in length.

Specific modifications in the CAAX tetrapeptide may be made which enhance the stability of the peptide with respect to resistance to proteolytic degradation, and which enhance the efficiency with which the peptide is taken up by cells. Such modifications include, but are not limited to, the synthesis of pseudopeptides, wherein amide bonds are reduced to secondary amines; the synthesis of carbapeptides, wherein amide nitrogens are replaced by carbon atoms; and the synthesis of azapeptides, wherein α-carbons are replaced with nitrogen atoms. Peptides having any and all such modifications should be construed to be included in the methods present invention provided the modified peptide inhibits the prenylation of an oncogene protein product in the assays described herein.

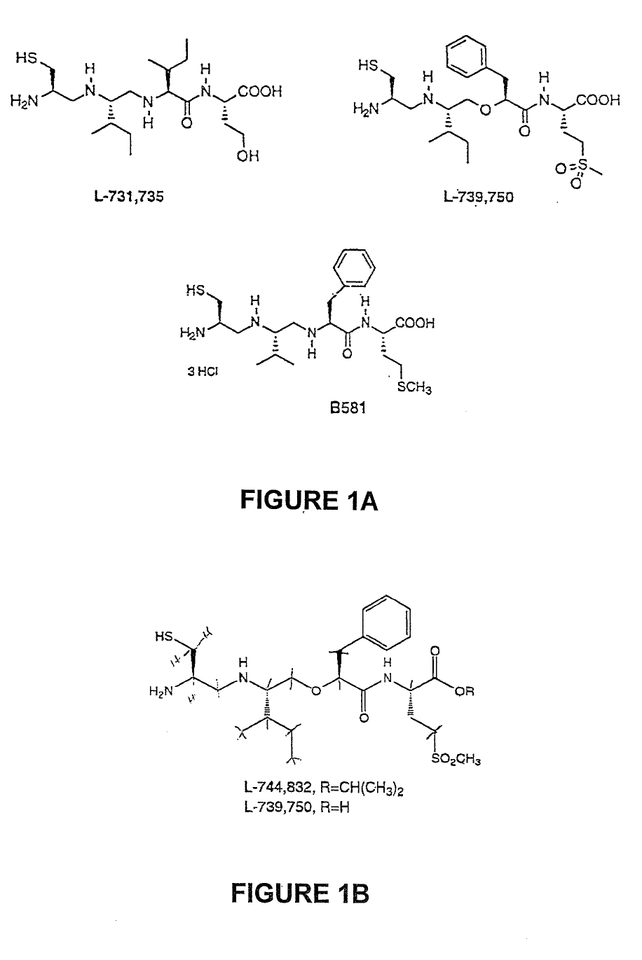

Examples of modified CAAX peptides include, but are not limited to, the peptidometic L-731,735 which is a CVLS pseudopeptide, wherein the first two peptide bonds are reduced as shown in FIG. 1A (Kohl et al., 1993, Science 260:1934-1937; Graham et al., 1994, J. Med. Chem. 37:725-732). Similarly, a corresponding CVFM pseudopeptide, named B581, also shown in FIG. 1A, has been described (Garcia et al., 1993, J. Biol. Chem. 268:18415-18418). Another peptidometic, named L-739,750 and also shown in FIG. 1A, is a modified peptide isostere where a methyleneoxy group has been replaced by an amide bond. This compound and methyl ester thereof, named L-739,749, are very potent inhibitors of FTase in vitro (Kohl et al., 1995, J. Cell. Biochem. 22:145-150). Similarly, B581 and its methyl ester also inhibit FTase (Garcia et al., 1993, supra). As will be described herein in the Experimental Details section, another compound L-744,832 which is shown in FIG. 1B and is similar in structure to the compounds shown in FIG. 1A, is also a potent FTase inhibitor.

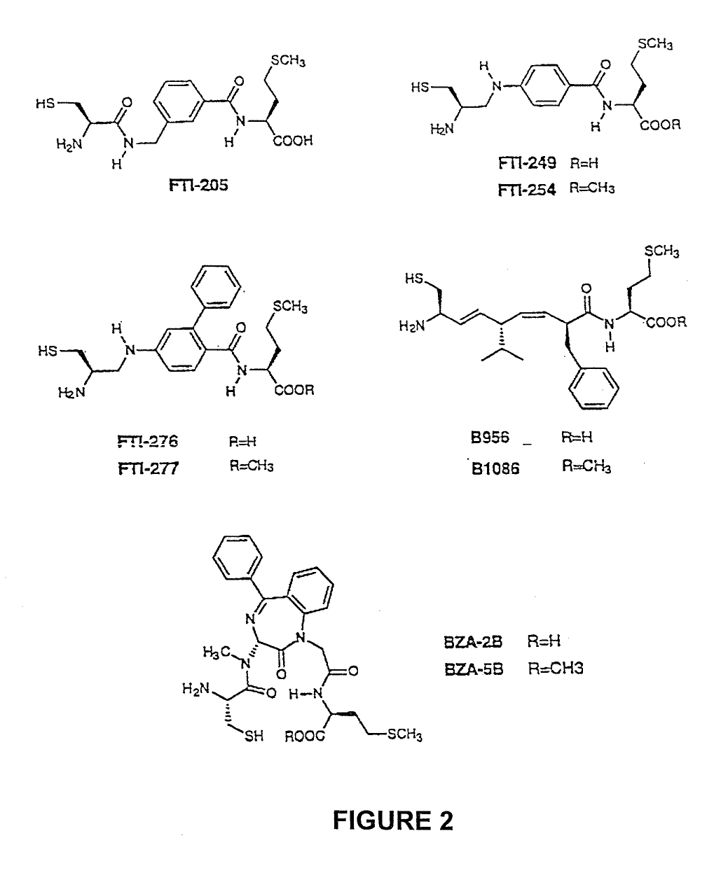

Using yet another approach for modification of tetrapeptides which are capable of inhibiting prenylation of proteins, the central two aliphatic amino acids may be replaced by hydrophobic dipeptide mimetics. This results in peptidometics having increased resistance to proteolytic degradation and also having enhanced cellular uptake properties. In one embodiment, the dipeptide “VI” in CVIM may be replaced by the simple dipeptide mimic 3-aminomethylbenzoic acid which separates cysteine and methionine. This results in the peptidometic FTI-205 (FIG. 2), which contains two rather than four amino acids and has no amide bonds of a peptidic nature (Nigam et al., 1993, J. Biol. Chem. 268:20695-20698). Despite these structural differences, FTI-205 retains potent FTase inhibitory activity (Nigam et al., 1993, supra). Although this molecule, similar to its parent tetrapeptide CVIM, is unable to inhibit farnesylation of ras protein in whole cells, systematic derivatization and reduction of the amide bond linking cysteine to the spacer 4-amindbenzoic acid gives rise to FTI-249, which is a potent FTase inhibitor. Further, the methyl ester of FTI-249, named FTI-254, which comprises a masked free carboxylate negative charge is also an FTase inhibitor (Qian et al., 1994, J. Biol. Chem. 269:12410-12413; Qian et al., 1994, Bioorg. Med. Chem. Lett. 4: 2579-2584).

The strategy employed to generate the above-described compounds involved hydrophobic replacement and was based on the hypothesis that within the CVIM binding site of FTase there is a hydrophobic pocket that complements the bulky side chains of dipeptide “VI”. Comparison of the structures of the parent peptide CVIM and its peptidometic FTI-249, clearly indicate that more binding energy may be gained by modifying the spacers to fully occupy the large hydrophobic “VI” binding pocket in FTase. This was accomplished by positioning different substituents on the aromatic ring of FTI-249. A simple aromatic substitution at the 2-position of 4-amino benzoic acid resulted in a dramatic improvement in inhibition of FTase activity. The peptidometic FTI-276, and its methyl ester FTI-277, shown in FIG. 2, inhibited Ftase activity (Lerner et al., 1995, J. Biol. Chem. 270:26802-26806; Lerner et al., 1995, J. Biol. Chem. 26770-26773).

A similar strategy for the generation of peptidometics comprising prenylation inhibitors may be used wherein the central two amino acids of a CAAX molecule may be replaced by benzyl-substituted alkane spacers (Harrington et al., 1994, Bioorg. Med. Chem. Lett. 4:2775-2780; Nagasu et al., 1995, Cancer Res. 55:5310-5314). Such peptidometics, including B956 and its methyl ester B1086 as shown in FIG. 2, are also capable of inhibiting FTase (Nagasu et al., 1995, supra).

Other modifications of the CAAX tetrapeptide useful in the methods of the invention include replacement of the central two amino acid residues in CAAX with 3-amino-1-carboxylmethyl-5 phenylbenzodiazepine-2-one (James et al., 1993, Science 60:1937-1942). The benzodiazepine peptidometic BZA-2B and its methyl ester BZA-5B shown in FIG. 2, have excellent FTase inhibitory activity (James et al., 1993, supra).

A key feature of the compounds described in FIGS. 1 and 2 is their high specificity for inhibition of FTase compared with GGTase.

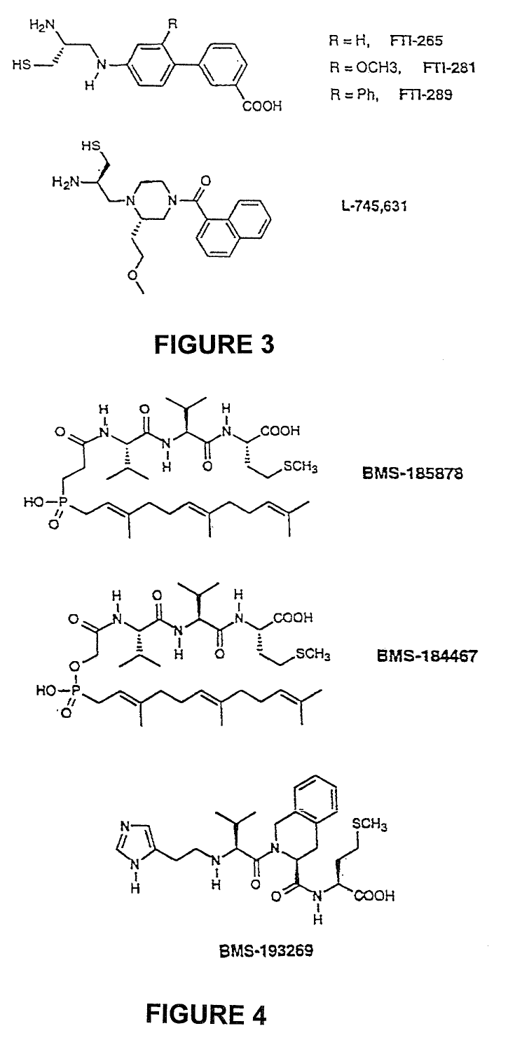

The peptidometics described thus far have peptide properties. The hydrophobic spacer strategy just described may be extended to include replacement of the methionine residue in order to obtain a true non-peptide peptidometic. This is accomplished by linking reduced cysteine to the tripeptide “VIM” mimetic, 4-amino-3-carboxybiphenyl. In FIG. 3, there is shown the peptidometic FTI-265, which contains no hydrolyzable bonds and no peptidic features, yet it retains potent FTase inhibiting activity (Vogt et al., 1995, J. Biol. Chem. 270:660-664). Furthermore, FTI-265 is highly specific for FTase compared with GGTase, despite the fact that this compound lacks the methionine residue which usually dictates specificity for GGTase (Vogt et al., 1995, supra). In addition, hydrophobic substitution at the 2 position of the first phenyl ring of the biphenyl moiety also results in increased enzyme binding affinity (Qian et al., 1996, J. Med. Chem. 39:217-223) and the substitution of methoxy or phenyl groups in this position (FIG. 3) increases the potency of FTase inhibition by 2 to 10-fold.

Also useful in the methods of the present invention is a series of non-peptide peptidometics wherein the “IIM” tripeptide terminus of CAAX is replaced by aryl substituted piperazines (FIG. 3). For example, the peptidometic L-745,631 inhibits FTase activity in whole cells. Interestingly this peptidometic is competitive with respect to H-Ras binding to FTase, despite major structural differences including the lack of the free carboxylate (Williams et al., 1996, J. Med. Chem. 39:1345-1348).

Taking advantage of characteristics of both the CAAX tetrapeptide and the isoprenoid farnesyl pyrophosphate, these characteristics may be incorporated into the design of FTase inhibitors. This rationale is based on the fact that an enzyme has highest affinity for its transition state. Several bisubstrate analogs where the tripeptide VVM is linked to the farnesyl group through spacers containing phosphinic or phosphonic acids, respectively, have been generated. These molecules are shown in FIG. 4. These molecules are highly selective for FTase as compared with GGTase (Bhide et al., 1994, Bioorg. Med. Chem. Lett. 4:2107-2112; Manne et al., 1995, Oncogene 10:1763-1779; Patel et al., 1995, J. Med. Chem. 38:435-442).

Several other FTase inhibitors have been synthesized which are useful in the methods of the present invention. For example, a KCA₁A₂X peptidometic has been generated wherein A₂ is replaced by conformationally constrained amino acid, (L)-1,2,3,4-tetrahydro-3-isoqunilinecarboxylic acid (Tic). One of their most potent compounds in this group of compounds is KCVTicM (Clerc et al., 1995, J. Bioorg. Med. Chem. Lett. 5:1779-1784). A family of peptidometics has been generated, including BMS-193269, wherein the cysteine has been successfully replaced by a non-thiol containing derivative such as imidazole. BMS-193269 (FIG. 4) is a potent inhibitor of FTase (Hunt et al., 1996, J. Med. Chem. 39:353-358). Further, a non-thiol-containing compound, (bz-(0)-His-Tyr-Ser (PD-15169) has been prepared which inhibits FTase activity (Sebolt-Leopold et al., 1995, 86th Annual Meeting of the American Association for Cancer Research, Toronto, Canada, Abstract #2561).

For each of the peptidometics described herein, the use of the term “FTase inhibition” should be construed to include inhibition of oncogene prenylation unless otherwise explicitly stated.

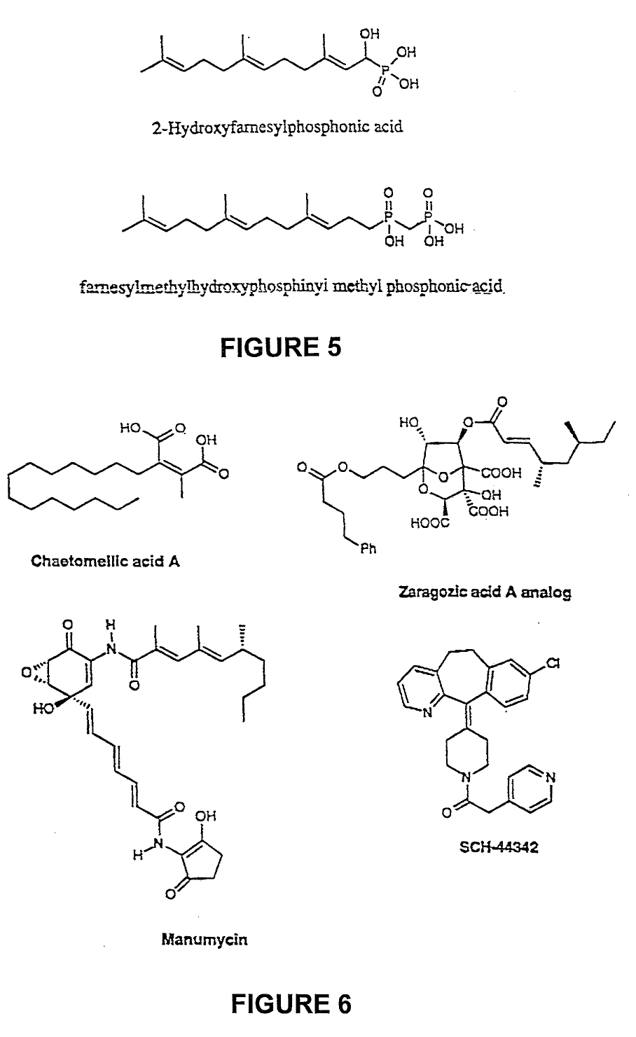

In addition to the peptidometics described herein, other farnesyl transferase inhibitors useful in the methods of the present invention include farnesyl pyrophosphate (FPP) analogs. FPP analogs have two limitations when used as inhibitors of FTase. They have a highly charged character because of the presence of the pyrophosphate and further, the inhibition of FTase thereby may extend to other FPP utilizing enzymes such as squalene synthase, which extension may be undesirable in the methods of the present invention. However, despite these limitations, there are several biologically active and selective FPP analogs which are useful in the methods of the invention. These include 2-hydroxyfarnesylphosphonic acid (Gibbs et al., 1993, J. Biol. Chem. 268:7617-7620) and farnesylmethyl-hydroxyphosphinylmethyl phosphoric acid, both of which are shown in FIG. 5 (Kothapalli et al., 1993, Lipids 28:969-973 Cohen et al., 1995, Biochem. Pharm. 49:839-845). Both of these compounds inhibit FTase. The α-hydroxyfarnesyl phosphonic acid also inhibits H-Ras processing (Gibbs et al., 1993, J. Biol. Chem. 268:7616-7620).

Natural products obtained from microorganisms, soils, or plants, as well as synthetic chemical libraries provide an immense pool of structures for random screening of FTase inhibition. These screens present a powerful means to obtain chemical compounds which may be modified using traditional medicinal chemistry for further drug development. Over fifteen FTase inhibitors from a variety of screens have been reported. The structures of some of these compounds are shown in FIG. 6. A more detailed discussion of these compounds is presented in Sattler et al. (1996, Mol. Biol. Intelligence Unit Series, Ed. Maruta, R. G. Landes, Austin, Tex.) and in Tamanoi (1993, Trends Biochem. Sci. 18:349-353). One particular compound, SCH-44342, possesses useful properties. This molecule is a non-peptide tricyclic inhibitor of FTase that contains no thiol or carboxylic acid groups (FIG. 6), yet it is a competitive inhibitor of FTase with respect to inhibition of ras protein prenylation (Bishop et al., 1995, J. Biol. Chem. 270:30611-30618).

Also included in the methods of the invention are compounds which inhibit the activity of GGTase, specifically GGTase I. GGTase I is capable of prenylation of a peptide having the sequence CAAX, whereas GGTase II requires the entire protein as a prenylation substrate. The substrate specificity of GGTase I is more stringent than that of FTase. However, as described herein, it is now known that K_{B}-ras, the most frequently mutated form of ras in human cancers, may be geranylgeranylated and that a GGTase I inhibitor, GGTI-286, blocks K_{B}-ras protein processing in K_{B}-ras oncogene-transformed NIH 3T3 cells (James et al., 1995, J. Biol. Chem. 270:6221-6226; Lerner et al, 1995, J. Biol. Chem. 270:26770-26773; Lerner et al., 1995, J. Biol. Chem. 270:26802-26806).

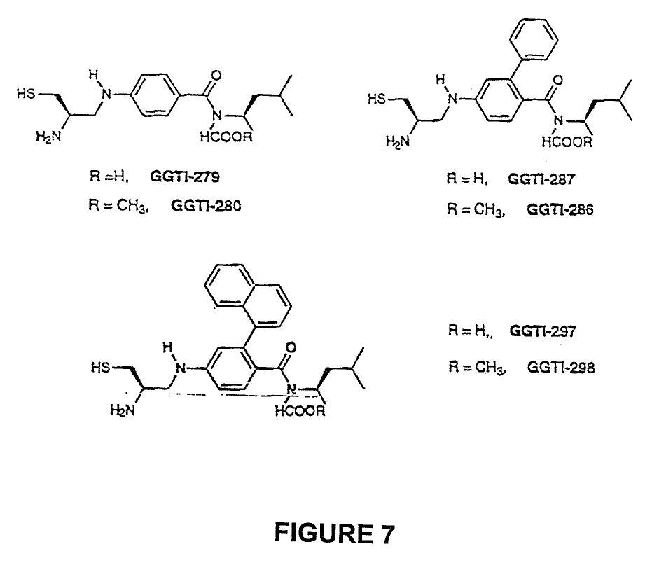

A GGTase I peptidometic inhibitor has been designed, named GGTI-279 (FIG. 6), which is a CVLL peptidometic wherein reduced cysteine and leucine were linked by-4-aminobenzoic acid spacers. GGTI-279 inhibited GGTase I preferentially over inhibition of FTase (Kauffman et al., 1995, Proc. Natl. Acad. Sci. USA 92:10919-10923). When the hydrophobicity of the spacer was increased, such as that shown in the compound GGTI-287 (FIG. 7), the potency of inhibition of GGTase I and FTase was increased 20-fold (Lerner et al., 1995, supra). The methyl ester compound, GGTI-286, was a potent inhibitor of posttranslational processing of Rap1A, an exclusive substrate for GGTase I, and also inhibited K_{B}-ras processing, but to a lesser extent that the inhibition observed with respect to Rap1A (Lerner et al., 1995, supra).

Linking reduced cysteine to methionine with 2-naphthyl 4-aminobenzoic acid resulted in GGTI-297 and its methyl ester GGTI-298 (FIG. 7) which are also inhibitors of GGTase I and FTase (Vogt et al., 1996, Oncogene, 13:1991-1999; McGuire et al., 1996, J. Biol. Chem. 271:27402-27407). GGTI-297 has two interesting properties. First of all, despite the fact that it is 10-fold less potent than GGTI-287, its methyl ester GGTI-298 is as potent as GGTI-286 with respect to inhibition of Rap1A processing in whole cells. This may be due to enhanced cellular uptake resulting from the more hydrophobic spacer (2-naphthyl vs 2-phenyl). Secondly, even though the selectivity of GGTI-297 for GGTase I over FTase is only 5-fold in vitro, its methyl ester, GGTI-298, at a concentration of 10 μM, is capable of completely inhibiting Rap1A protein processing without affecting H-ras processing.

In summary, a variety of FTase and GGTase I inhibitors are known in the art and are capable of inhibiting the activity of these enzymes in addition to inhibiting posttranslational modification of an oncogene protein product. The methods of the invention should therefore be construed to include any and all FTase and GGTase I inhibitors which inhibit prenylation of an oncogene protein and which render cells more sensitive to either radiation or chemotherapy or render cells more sensitive to both radiation and chemotherapy.

As will be described in more detail in the Experimental Details section provided herein, the inhibition of prenylation of an oncogene protein product results in increased radiation sensitivity of cells. As discussed herein, inhibition of prenylation of an oncogene protein product may also confer on cells increased sensitivity to chemotherapeutic agents when the chemotherapeutic agent relies on cellular DNA replication as the means by which it effects cell killing.

The methods of the invention should not be construed to be limited to the particular oncogene exemplified in the Experimental Details section, i.e., the H-ras or K-ras oncogenes. Rather, the invention should be construed to include any and all ras oncogenes wherein when the protein product of the oncogene is inhibited, tumor cells are more sensitive to radiation and/or chemotherapy. Thus, the oncogenes which are preferred in the methods of the invention are those which are involved in the ras signalling pathway.

The invention should also not be construed as being limited solely to oncogenes per se. Other protein products which participate in the ras signalling pathway are also included in the invention as targets for inhibition, preferably inhibition of protein prenylation, provided inhibition of the function of these other proteins results in enhanced sensitivity of cells to radiation and/or chemotherapy.

By the term “participation in the ras signalling pathway” as used herein, is meant a protein which is an essential component in the ras signalling pathway.

In particular with respect to prenylation inhibitors, oncogenes protein products and other proteins which are useful in the methods of the invention include those proteins having a CAAX sequence at the carboxyl terminus and wherein the inhibition of prenylation thereof results in increased sensitivity of cells to radiation and/or chemotherapy. Oncogenes which are useful in the present invention include, but are not limited to, each of the ras proteins such as H, K_{A}, K_{B} and N-ras. In addition, as noted herein, the invention should be construed to include other proteins which participate in the ras signalling pathway leading to radiation resistance of cells. These proteins include, but are not limited to, rhoA, rhoB, rhoC and RAC-1, each of which is prenylated.

Tumor cells may be tested for the presence of a desired oncogene protein product using any number of immunochemical techniques, including, for example, Western blotting. Tumor cells may be further tested for the presence of prenylated forms of the oncogene protein also using Western blotting. Once it is known that a tumor contains cells which express an oncogene protein product which is prenylated, then the cells in the tumor are candidate target cells for the use of prenylation inhibitors for conferring radiation and/or chemotherapy sensitivity on the cell.

By the term “conferring radiation sensitivity on cells” as used herein with respect to the properties of a particular compound, is meant that cells are rendered more sensitive to the effects of radiation in the presence of the compound than in the absence of the compound.

To determine which combinations of oncogenes and prenylation inhibitors confer radiation and/or chemotherapy sensitivity to cells, the following procedures may be used. A combination of oncogene and known or putative inhibitor may be tested for (i) the ability to inhibit prenylation of the oncogene protein product and, (ii) for the ability to increase radiation sensitivity and/or chemotherapy of cells. The details of such tests are described herein in the Experimental Details section.

Essentially, a suitable population of cells is transfected with DNA comprising the oncogene. A prenylation inhibitor is added to the cells either concomitantly with the DNA, or is added to the cells either prior to or following the addition of DNA. Prenylation of the subject oncogene, or the lack thereof, may be assessed by immunochemical means, such as Western blotting and the like. The sensitivity of the cells to radiation treatment may be assessed in an apoptosis assay, a cell survival assay and the like, as described in the Experimental Details section. The sensitivity of cells to chemotherapy may be assessed using similar methodology to that used for assessment of radiation sensitivity of cells. In addition, the sensitivity of cells to chemotherapy may be assessed using any of the protocols described in Carmichael et al. (1987, Cancer Res. 47:936-942).

The identification of oncogenes whose protein products may be manipulated by prenylation inhibitors is important to the discovery of the types of tumors against which the prenylation inhibitor will be effective. Oncogene transfected cells which are administered a prenylation inhibitor and wherein prenylation of the oncogene protein product is inhibited, are then tested for their relative sensitivity to radiation and chemotherapy and the results are compared with those obtained in similarly treated cells which are either not transfected or have not been administered the prenylation inhibitor. In this manner, cells may be identified which because they express a particular oncogene, are suitable candidates for treatment with an appropriate prenylation inhibitor in order to increase their sensitivity to radiation and/or chemotherapy.

The methods of the invention are not limited to the use of a single protein product inhibitor as a means of conferring radiation sensitivity on cells. Rather, the methods of the invention may include the use of one or more protein product inhibitors as a means of conferring radiation sensitivity on cells. The types of combinations of inhibitors which may be used for this purpose may be identified using the procedures and assays described herein.

As will be apparent from the data presented herein, the methods of the invention are applicable to several different types of tumors in animals including, but not limited to, solid tumors such as tumors of the prostate, lung, colon, breast, pancreas, cervical carcinoma or sarcoma, rectal tumors, ovarian tumors, bladder and thyroid tumors and head and neck tumors. Tumors which are most preferably treated using the methods of the invention include tumors of the pancreas, lung and colo-rectal tumors.

Animals which are administered ras protein inhibitors are either irradiated or are administered chemotherapy in conjunction with the administration of the inhibitor. It will be appreciated that the precise protocols to be used for administration of either radiation or chemotherapy to a tumor bearing animal will depend on any number of factors including the age of the animal and the type of tumor to be treated. However, it will also be appreciated that one of skill in the art of treating tumors will know the precise protocols to be used once in possession of the present invention, the patient's tumor status and the patient's age, etc.

When irradiating an animal, generally, multiple doses of radiation are administered to the animal over a period of time in order that skin damage in the animal is minimized and the effect of the radiation on tumor growth in the animal is maximized. The rationale and methods involved in a multiple dose type radiation protocol is described in Hall (1994, Radiobiology for the Radiologist; Time, Dose and Fractionation in Radiotherapy, pp212-229, J. B Lippincott Company, Philadelphia, Pa.) and may be used in the present invention.

Protocols for the administration of chemotherapy to a tumor bearing animal are also described in Hall (1994, Radiobiology for the Radiologist; Chemotherapeutic Agents from the Perspective of the Radiation Biologist, pp.289, J. B Lippincott Company, Philadelphia, Pa.) which methods may be included in the methods of the present invention. In addition, if it is preferable to target chemotherapy to specific tumor sites in an animal, for example, by antibody tagging and the like, such methods of targetting chemotherapy should also be construed as being included within the methods of the present invention to effect reduction in growth or elimination of the tumor in the animal.

In the case of specific tumor types, the protocols for irradiation may be altered to suit the specific type of tumor being treated. For example, protocols for irradiation and chemotherapy of an animal having a colorectal tumor are described in Mohiuddin et al. (1991, Seminars, Oncology 18:411-419). Protocols for irradiation and chemotherapy of an animal having a sarcoma are described in Delaney et al. (1991, Oncology 5:105-118). It should be noted that in the latter instance, radiation is the preferred treatment for sarcoma. Protocols for irradiation and chemotherapy of an animal having a breast tumor are described in Mansfield et al. (1991, Seminars Oncology 18:525-535) and in Levitt (1994, Cancer 74:1840-1846). Protocols for irradiation and chemotherapy of an animal having a head or neck tumor are described in Harari et al. (1995, Curr. Opin. in Oncol. 7:248-254). Protocols for treatment of cervical tumors are described in Perez (1993, Oncology 7:89-96) and protocols for treatment of prostate tumors are described in Perez et al. (1993, Cancer 72:3156-3173).

Preferably, the animal which is treated is a human.

The preferred prenylation inhibitors useful in the invention are FTI-276, FTI-277, GGTI-297 and GGTI-298. These inhibitors may be rendered even more useful in the methods of the invention when the sulfhydryl groups thereon are removed such that the biological half lives of the inhibitors is extended. The methods for removal of sulfhydral groups from these compounds are well know to the skilled chemist working in the field of prenylation inhibitors.

The preferred oncogenes against which the prenylation inhibitor is directed are the ras proteins.

The precise route of administration of an oncogene protein prenylation inhibitor and the amount, and frequency of administration of the inhibitor which is administered to an animal, preferably, a human, will depend on any number of factors, including, but not limited to, the location of the tumor, the age of the animal and the severity of the disease. It will be appreciated that the precise route of administration, the frequency of administration and the dose administered will be apparent to the artisan skilled in the art of administering such compounds to cancer patients.

In general, a prenylation inhibitor may be administered to an animal in one of the traditional modes (e.g., orally, parenterally, transdermally or transmucosally), in a sustained release formulation using a biodegradable biocompatible polymer, or by on-site delivery using micelles, gels and liposomes, or rectally (e.g., by suppository or enema) or nasally (e.g., by nasal spray). The appropriate pharmaceutically acceptable carrier, salts solution, and the like, will be evident to those skilled in the art and will depend in large part upon the route of administration.

Treatment regimes which are contemplated include a single dose or dosage which is administered hourly, daily, weekly or monthly, or yearly. Dosages may vary from 1 μg to 1000 mg/kg of body weight of the inhibitor and will be in a form suitable for delivery of the compound.

The route of administration of the inhibitor may also vary depending upon the disorder to be treated. The invention contemplates administration of an inhibitor to an animal for the purpose of treating cancer in the animal. One protocol for administration of a prenylation inhibitor to a human is provided as an example of how to administer a prenylation inhibitor to a human. This protocol should not be construed as being the only protocol which can be used, but rather, should be construed merely as an example of the same. Other protocols will become apparent to those skilled in the art when in possession of the present invention. Essentially, for administration to humans, the inhibitor is dissolved in about 1 ml of saline and doses of 1 μg, 10 μg, 100 μg or even several milligrams per kg of body weight are administered intravenously at 48 hour intervals.

An animal having a tumor which has been administered the prenylation inhibitor is then irradiated or is administered chemotherapy following the protocols for irradiation of an animal or administration of chemotherapy to an animal as described herein.

The invention also includes a method of identifying a prenylation inhibitor which confers radiation or chemotherapy sensitivity on a cell population. The method comprises providing a population of cells which express a protein product which participates in the ras signalling pathway and which is in need of prenylation for its activity. A test compound is added to the cells which are also irradiated or are treated with a chemotherapy agent. The level of sensitivity of the cells to irradiation or chemotherapy is then assessed. A higher level of sensitivity of the cells to radiation or chemotherapy in cells administered the test compound compared with the level of radiation or chemotherapy sensitivity in cells which were not administered the test compound, is an indication that the test compound confers radiation or chemotherapy sensitivity on the cell population. Assessment of radiation and or chemotherapy sensitivity of cells may be accomplished using the methods described herein in the Experimental Details section and those described in Carmichael et al. (1987, supra). For example, the sensitivity of the cells to radiation or chemotherapy may be assessed by measuring the extent of apoptosis of the cell population, or, simple cell survival assays may be performed.

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Experimental Details

### EXAMPLE 1

### Radiosensitization of Rodent Cells Using Prenyltransferase Inhibitors

Cells

The oncogene-transfected cells used in this study were all derived from early passage rat embryo fibroblast (REF) by transfection with the pEJ plasmid containing the H-ras gene isolated from the EJ bladder carcinoma. This vector was introduced by calcium phosphate DNA transfer into primary REF, either alone or together with the pMC29 vector containing v-myc. One clone containing both the introduced H-ras gene and the v-myc genes is 3.7 (McKenna et al., 1990, Cancer Res. 50:97-102). 4R and 5R cells were obtained as rare transformants after transfection with the pEJ plasmid alone (McKenna et al., 1993, supra). MR4 cells were immortalized by transfection with an expression vector comprising v-myc linked to a neomycin-resistant selectable marker (McKenna et al., 1991, Radiat. Res. 125:283-287). REF-GG cells were obtained by transforming REF cells with a chimeric H-ras(v12) in which the CAAX motif is CVLL. All cell lines were mycoplasma free.

Radiation Survival Determination

Cells obtained from log growth cultures were plated in 60-mm dishes and allowed to attach for 4 hours before addition of FTI-277 to the medium. The plating medium was then removed and fresh medium containing FTI-277 in DMSO or DMSO alone (for control dishes) was added immediately before irradiation. Cells were irradiated with a Mark I cesium irradiator (J. S. Shepherd, San Fernando, Calif.) at a dose rate of 1.7 Gray per minute. Colonies of cells were stained and counted 10-14 days after irradiation (McKenna et al., 1990, supra). The surviving fraction of cells at a given dose is defined as:

$$\frac{Number of colonies formed}{( Number of cells plated ) ( Plating efficiency )}$$

Each point on the survival curves represents the mean surviving fraction from at least three dishes of cells.

Apoptosis Determination

3×10³ cells/well in 24-well plates were allowed to attach for 12 hours and were then treated with medium containing FTI-277 in DMSO or an equivalent volume of DMSO without inhibitor. Twenty four hours after irradiation with 10 Gray using the irradiator described above, both adherent and non-adherent cells were harvested. Cells were stained with propidium iodide (Muschel et al., 1995, Cancer Res. 55:995-998). A minimum of three independent fields of 100 cells were counted for each sample. Scoring of samples was performed in a double blind manner.

Western Blotting

Cells were lysed in culture dishes with 1 x reducing Laemmli sample buffer. Samples were boiled, sheared and clarified by centrifugation at 14,000 rpm in a microfuge before storage at −20° C. Samples containing equal amounts of protein were separated on a 12% SDS polyacrylamide gel and electrophoretically transferred to nitrocellulose membranes. Membranes were blocked for 1-2 hours in PBS containing 0.1% Tween-20 and 5% powdered milk before antibody addition. Membranes were probed with monoclonal pan-ras antibody AB-4 (Oncogene Science, Uniondale, N.Y.) at a concentration of 0.5 μg/ml, or with monoclonal H-ras antibody LA069 (1:5000 dilution; Quality Biotech, Camden, N.J.). Detection of protein was accomplished using the ECL chemiluminescence kit (Amersham, Arlington Heights, Ill.). Images were digitized using an Arcus II scanner, and figures were assembled using Adobe Photoshop 3.0.

The results of the experiments presented in Example 1 are now described.

Inhibition of Prenylation of H-ras after FTI-277 Treatment of REF Cells Transformed by H-ras

Transformed REF cells derived from the R5 cell line were treated with 5 μm FTI-277 to establish a time course for the accumulation of unfarnesylated H-ras. Prenylated forms of ras migrate more rapidly than unprenylated ras on SDSpolyacrylamide gels (Gutierrez et al., 1989, EMBO J. 8:1093-1098; Farh et al., 1995, Arch. Biochem. Biophys. 18:113-121). This shift in motility was used to assess the prenylation status of H-ras after farnesyltransferase inhibitor treatment.

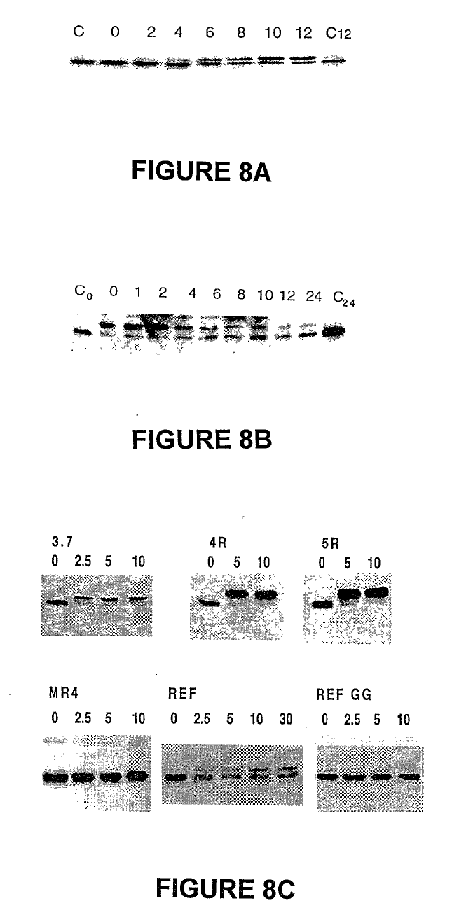

Samples were harvested at various time points and the proteins present were identified by Western blot analysis using an anti-H-ras antibody (FIG. 8A). The conversion of H-ras to the slower moving form was observed by 4 hours after the addition of FTI-277 to the cells. By 6-8 hours after the addition of the inhibitor, approximately 50% of the detectable H-ras was in the unfarnesylated form. The same results were obtained when the 3.7 REF cell line was similarly treated.

The reversibility of FTI-277 inhibition was also examined. Cells derived from the 3.7 REF cell line were treated with 5 μm FTI-277 for 30 hours. The inhibitor was then removed and the reappearance of farnesylated H-ras in the cells was examined (FIG. 8B). Detectable amounts of farnesylated H-ras were observed by 2 hours and increase to about 50% of H-ras being in the farnesylated form by 6 hours. By 24 hours, the majority of the H-ras in the cells was in the farnesylated form. Therefore, the accumulation of unfarnesylated H-ras occurs rapidly in H-ras transformed REF cells, and is reversed within 24 hours after removal of the inhibitor.

The specificity inhibition of H-ras^{v12} farnesylation by FTI-277 was examined by comparing the effect of this inhibitor on a panel of REF cells transformed with ras. Exposure of H-ras oncogene transformed REF cells (3.7, 4R, and 5R) to various dose of FTI-277 (2.5 to 10 μm) for 24 hours resulted in H-ras proteins which were primarily unfarnesylated (FIG. 8C). However, cells which expressed wild type c-H-ras were less susceptible to the effects of this inhibitor. Treatment of untransformed REF resulted in detectable H-ras in the slower moving, unfarnesylated form, although the majority of the H-ras remained in the farnesylated form in spite of treatment of the cells with doses of up to 30 μM of inhibitor. MR4 cells (REF immortalized with v-myc) appear to express very low levels of H-ras (ras protein was only detectable in these cells using a pan-ras antibody) and no changes in the migration of ras were observed with treatment of up to 10 μm of FTI-277.

Inhibition of Prenylation of H-ras Following Treatment of H-ras Transformed REF Cells with the Inhibitor, L-744,832

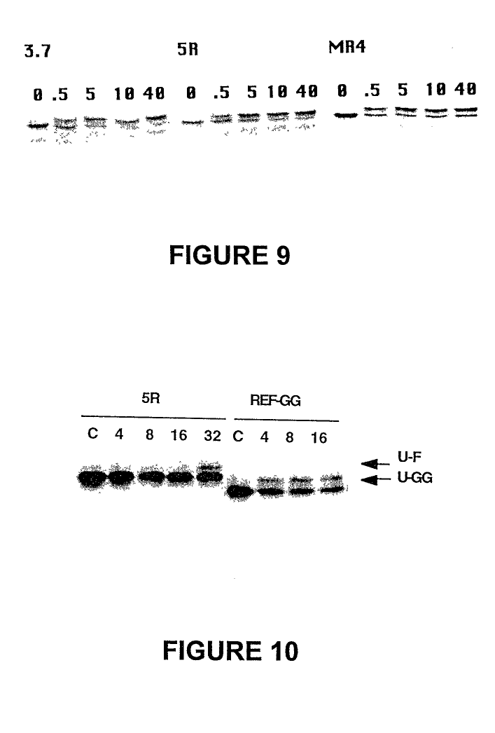

Another farnesylation inhibitor, L-744,832 whose structure is shown in FIG. 1B, was tested for the ability to inhibit farnesylation of ras in REF cells transformed with ras. The results which were obtained with this inhibitor were similar to those obtained using the inhibitor FTI-227 (FIG. 9). Cells in log phase culture were treated with varying doses of the inhibitor L-744,832. Twenty four hour treatment with this inhibitor resulted in an accumulation of a majority of H-ras in the unfarnesylated form at a dose of between 0.5 μm and 5 μm in 3.7 and SR cells. The endogenous H-ras in MR4 cells was found to be less susceptible to farnesylation inhibition in that farnesylated form of this protein was observed to persist in these cells at the highest dose of inhibitor used.

Inhibition of Geranylgeranylation Using the Inhibitor GGTI-286

The targeting of prenyltransferases to a particular ras protein is in large part dictated by the CAAX recognition sequence found at the carboxyl terminal portion of ras and other prenylated proteins (Reiss et al., 1991, Proc. Natl. Acad. Sci. USA. 88:732-736). The cysteine within the carboxyl terminal end of H-ras, CVLS, is the target for prenylation by farnesyltransferase. In contrast, CAAX sequences terminating in leucine have a greatly reduced affinity for farnesyltransferase and are instead geranylgeranylated by GGTase I (Cox et al., 1992, Mol. Cell. Biol. 12:2606-2615). A chimeric H-ras^{v12} with CVLL as the CAAX motif is capable of transforming NIH 3T3 cells and Rat-1 cells. This altered H-ras also transforms primary REF in when co-transfected with v-myc (Bernhard et al., 1996, Cancer Res. 56:1727-1730).

Compared with 3.7 or 5R, REF cells transformed with H-ras^{v12} CVLL (REF-GG) adhere poorly to tissue culture dishes and do not form discrete colonies. These cells serve as useful controls in the experiments described herein since the H-ras^{v12} CVLL protein should be impervious to the effects of FTI-277, but should be sensitive to GGTI-286 mediated inhibition of GGTase I. When REF-GG cells were treated with up to 10 μM FTI-277, no change in the mobility of H-ras-CVLL was observed (FIG. 8C). These results establish that FTI-277 is specific for FTase rather than for GGTase I. Since the apoptosis and survival experiments reported below were performed using FTI-277 at doses below 10 μM, these results were obtained at concentrations of this inhibitor which do not affect GGTase I activity.

In human tumor cell lines, the inhibitor, FTI-277, largely affects posttranslational processing of H-ras rather than K-ras. Two possible explanations may account for this finding. The first is that FTI-277 is a competitive inhibitor of FTase. Since FTase has a seven-fold higher affinity for the K-ras CAAX sequence than it has for the H-ras sequence (Reiss et al., 1991, supra), FTI-277 inhibition of K-ras farnesylation is expected to be less efficient than the inhibition of H-ras farnesylation by this compound. Secondly, cross prenylation of K-ras by GGTase I may occur when farnesylation is inhibited (James et al., 1995, J. Biol. Chem. 270:6221-6226). To deal with the possibility that some K-ras is modified through geranylgeranylation, studies were initiated to assess the effects of novel geranylgeranyltransferase inhibitors, GGTI-286 (Lerner et al., 1995, J. Biol. Chem. 270:26770-26773) and a more effective inhibitor, GGTI-298 (McGuire et al., 1996, J. Biol. Chem. 271:27402-27407; Vogt et al., 1996, Oncogene 13:1991-1999), on K-ras processing. Preliminary results using the inhibitor GGTI-286 demonstrated effective inhibition of the geranylgeranylation of the chimeric H-ras^{v12} having the CVLL recognition sequence for GGTase I expressed by REF-GG cells at inhibitor doses as low as 4 μM. In contrast, the H-ras^{v12} expressed by the 5R cell line which has the farnesyltransferase recognition sequence CVLS, and is thus farnesylated, is only partially inhibited by this inhibitor at a dose of 32 μM (FIG. 10). The endogenous wild-type c-H-ras in the MR4 cell line is expressed at very low levels, but no effect on the migration of this protein by GGTI-286 treatment was observed. These data demonstrate that GGTase I inhibitors are effective and specific inhibitors of geranylgeranylation.

Given these data, it is now possible to test whether FTase inhibitors used in conjunction with GGTase I inhibitors more effective in inhibiting K-ras prenylation than either compound alone. Even if K-ras is a substrate for both farnesyltransferase and geranylgeranyltransferase I, when both types of inhibitors are used together, inhibition of prenylation of oncogene proteins will be facilitated.

Morphological Alterations Induced in Cells by FTI-277

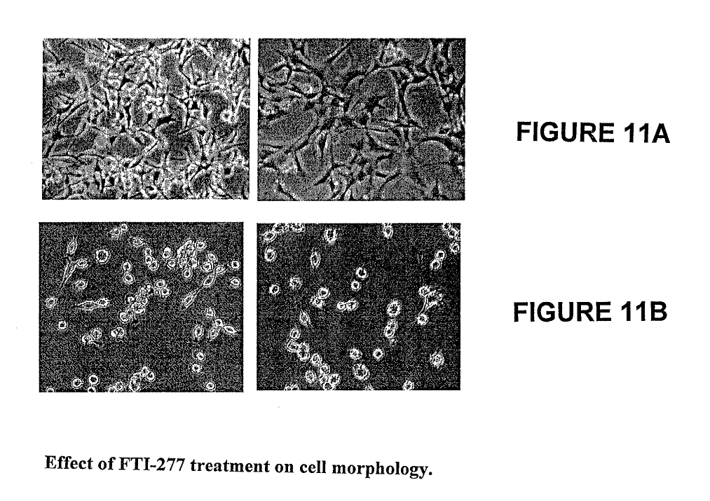

The treatment of H-ras transformed REF cells with FTI-277 results in partial reversion of transformation of the cells (FIG. 11A). 3.7 cells which were treated with 2.5 μM FTI-277 became less refractile and increasingly spread out. 5R cells also became more flattened and had fewer processes, but the change was less pronounced in these cells than in 3.7 cells. The time course of the morphological changes induced by this inhibitor over 48 hours was considerably longer than the time course of the changes in H-ras mobility detected by Western blotting. This may be indicative of the presence of other proteins which are involved in the morphological reversion of ras transformed cells. However, treatment of REF-GG cells with 5 μM FTI-277 did not induce altered cell morphology (FIG. 11B), indicating that the effect of this inhibitor on cell morphology is influenced by the activity of this compound on ras prenylation.

Effect of FTI-277 Treatment on Apoptosis after Irradiation

Exposure of REF cells immortalized with v-myc to ionizing radiation resulted in high levels of apoptosis, while cells transformed with H-ras plus v-myc exhibit substantially lower levels of apoptosis. These results establish that transformed cells expressing activated H-ras which are more resistant to killing by irradiation are also more resistant to the induction of apoptosis by radiation than cells which do not express H-ras. These results also imply that the absence of H-ras activity in these cells leads to increased radiation-induced apoptosis and decreased clonogenic survival. As a first test of this prediction, the effect of the inhibition of H-ras farnesylation on apoptosis after irradiation was examined.

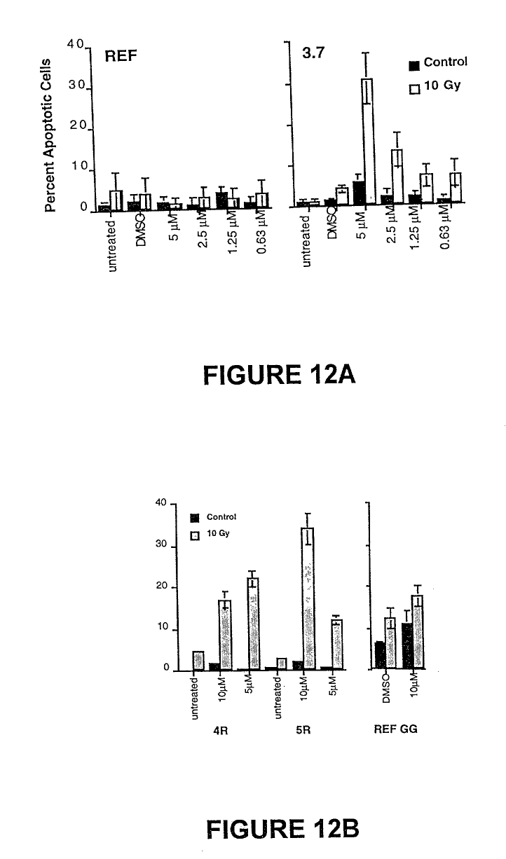

Cells were irradiated with 10 Gray and were treated concurrently with various concentrations of FTI-277. The extent of apoptosis induced by the irradiation of 3.7 cells, which express activated H-ras and v-myc, was greatly enhanced by treatment with FTI-277 (FIG. 12A). The maximum effect of the inhibitor on these cells was observed at an inhibitor concentration of 5 μM. However, a significant increase was also observed at a concentration of 2.5 μM of the inhibitor. Thus, doses of FTase inhibitor which inhibit H-ras^{v12} farnesylation cause an increase in the level of apoptosis in the cell population following irradiation of the cells. The observed increase in apoptosis levels is specific for cells which express activated H-ras since FTI-277 treatment of REF cells which are untransformed, did not result in an increase in apoptosis of these cells following irradiation.

The ability of FTI-277 to augment irradiation-induced apoptosis in cells transformed by the H-ras oncogene was confirmed in 4R and 5R cells (FIG. 12B). These cells are two independent clones of REF cells transformed with H-ras^{v12} alone. These results demonstrate that the presence of an activated H-ras alone is sufficient to cause increased radiation-induced apoptosis following FTI-277 treatment. The results shown in FIGS. 12A and 12B were obtained 24 hours after treatment; equivalent results were observed at 48 hours following treatment with the exception that some toxicity of the inhibitor became apparent at the highest doses in 3.7 cells only.

As an additional control, to examine the specificity of apoptosis induction following irradiation the REF-GG cell line was used. H-ras^{v12} expressed by these cells is not affected by FTI-277 treatment. Thus, the level of apoptosis after irradiation should not be increased in these cells when they are treated with FTI-277. REF-GG cells exhibited a relatively high baseline level of apoptosis of about 6% (FIG. 12B). This level was increased to 12% by irradiation. Treatment of these cells with FTI-277 slightly increased the baseline level of apoptosis, but had no significant effect on enhancing the extent of apoptosis after irradiation. Thus, the increase in apoptosis seen after irradiation and FTI-277 treatment appears to be specific to cells with oncogenic H-ras that is processed by the addition of a farnesyl group.

Inhibition of Ras Farnesylation and Effect on Radiation Induced Apoptosis in Mouse Prostate Tumor Cells Transformed with H-ras^{v12}

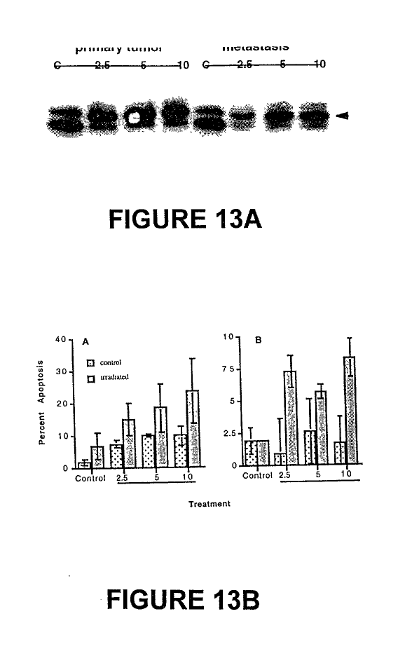

The observations obtained in the REF model system were extended to mouse prostate tumor cells derived by transduction of H-ras and v-myc into mouse embryo urogenital sinus cells (Thompson et al., 1989, Cell 56:917-930; Thompson et al., 1993, Cancer 71: S1165-S1171). Treatment of either primary tumor cells or a metastatic clone of this tumor line exhibited a dose-dependent reduction of the farnesylated form and the accumulation of the more slowly migrating, unprocessed form of H-ras (FIG. 13A). Thus, FTI-277 is an effective inhibitor of ras farnesylation in transformed prostatic epithelial cells.

When the prostatic epithelial cells were examined for radiation-induced apoptosis after treatment with FTI-277 at doses which inhibited ras farnesylation, a significant increase in apoptosis was observed (FIG. 13B). Thus, inhibition of farnesylation in prostate cells as well as fibroblasts resulted in increased radiation-induced apoptosis.

Decreased Radiation Survival of Ras Transformed REF Cells Treated with FTI-277

H-ras transformed REF cells are significantly more radioresistant than untransformed REF cells. Further, REF cells which are immortalized with myc are not altered in their ability to resist radiation when compared with parental REF cells. Therefore, inhibition of H-ras activity using the farnesylation inhibitor might be expected to reduce radiation resistance in cells expressing oncogenic H-ras.

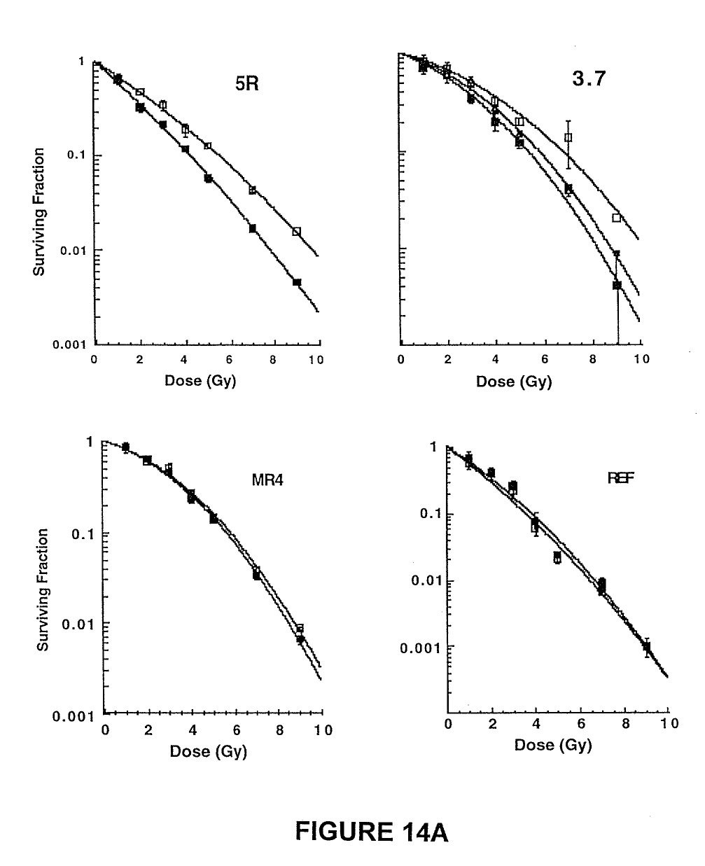

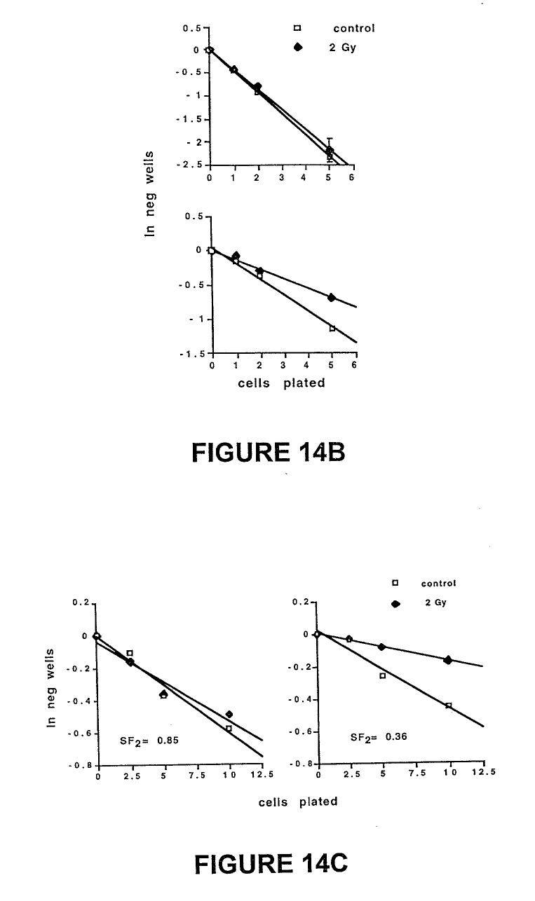

Cells were irradiated with the indicated doses of ionizing radiation and were treated with FTI-277 for 24 hours after irradiation (FIG. 14A). The survival curves for 3.7 and 5R cells demonstrated that these cells were more resistant to radiation than were MR4 or REF cells, MR4 cells being slightly more resistant to radiation than were REF cells. After treatment with FTI-277, the radioresistance of 3.7 and 5R cells was reduced to a level of survival which was similar to that seen in MR4 cells, the myc immortalized REF cells, or REF cells themselves. The shoulders of the survival curves were reduced as were the overall slopes of the survival curves. Exposure of the cell line MR4 or REF to FTI-277 had no effect on radiation survival. These results indicate that FTI-277 can act as a specific radiosensitizer of cells expressing an activated H-ras oncogene, but that the inhibitor has no effect on non-ras expressing cells. Because of their loose adherence and inability to form colonies, REF-GG cells could not be tested in standard clonogenic survival assays. The SF₂ (i.e., the fraction of clonogenic cells surviving irradiation at a dose of 2 Gray) for this cell line was determined by limiting dilution analysis of clonogens (Thilly et al., 1980, Serres et al., (eds.), Chemical Mutagens, Vol. 6, pp. 331-364. New York: Plenum; Grenman et al., 1989, Int. J. Cancer 44:131-136) arising after 2 Gray irradiation in the presence of 5 μM FTI-277 or 8 μM GGTI-298. In this assay, survival of one or more cells resulted in colony formation in a well, and secondary colonies no longer complicate the scoring of clonogenic survival since the frequency of negative wells is scored, and survival is derived from Poissonian statistical analysis.

Irradiation with 2 Gray after treatment with drug carrier alone resulted in an SF₂ of 0.91 (FIG. 14B, top). Treatment with 5 μM FTI-277 resulted in a slight decrease in survival to an SF of 0.81. In contrast, treatment of REF-GG cells with 8 μM GGTI-298 reduced the SF₂ to 0.64 (FIG. 14B, bottom), demonstrating that inhibition of ras prenylation would also reduce radiation resistance when ras was modified by geranylgeranyltransferase.

Radiosensitization of murine prostate tumor cells by FTI-277 treatment was also observed. As shown in FIG. 14C, survival after 2 Gray irradiation of H-ras plus v-myc transformed mouse prostate tumor cells was reduced from 0.85 to 0.36. This demonstrates that radiosensitization can be obtained not only in sarcomas, which are of mesenchymal origin, such as the fibroblast derived 3.7 and 5R tumor cells, but in tumors of endothelial origin such as prostate tumors.

Effect of FTI-277 Treatment on REF Cell Growth

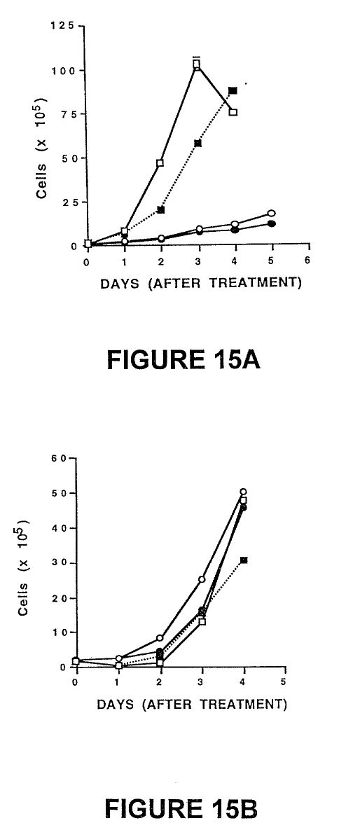

Farnesyltransferase inhibition is known to affect cell growth in a manner that appears to be independent of ras status (Sepp-Lorenzino et al., 1995, Cancer Res. 55:5302-5309). In order to confirm that the results obtained in clonogenic survival assays were not simply due to complete growth inhibition, the growth of the myc+ras transformed 3.7 cells and the myc immortalized MR4 cell line was examined in the presence and absence of FTI-277 (FIG. 15A). The results demonstrate that FTI-277 treatment resulted in some inhibition of cell growth in 3.7 cells under the conditions used in radiation survival experiments; however, a fifty-fold increase in cell number was seen by day 4 after drug treatment. Similar results were obtained following FTI-277 treatment of the 5R cell line (FIG. 15B), which appeared more resistant to growth inhibition. These data together with the plating efficiencies obtained in clonogenic survival experiments demonstrate that the radiosensitization demonstrated in H-ras transfected REF cells is not an artifact of reduced cell growth in the presence of FTI-277.

### EXAMPLE 2

### Radiosensitization of Human Cells with Prenyltransferase Inhibitors

Inhibition of Prenylation in H-ras after FTI-277 Treatment of Human T24 Cells

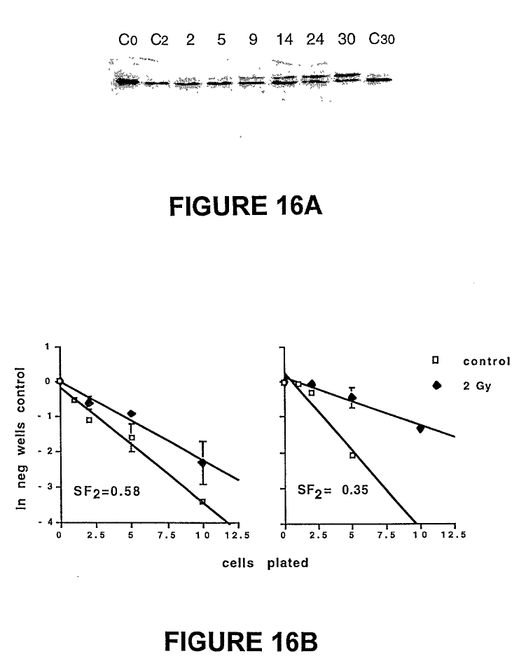

To extend the findings described in Example 1 to human cells, a panel of human tumor cell lines having naturally occurring ras mutations were examined. The effect of FTI-277 on the human bladder carcinoma cell line, T24, was first examined. These cells are the cells from which the H-ras^{v12} oncogene was first isolated. The time-course of accumulation of unfarnesylated H-ras was slower in this and other human cell lines than in REF cells (FIG. 16A). This is likely due to the slower rate of synthesis of H-ras in these cells, rather than to turnover in human cells since the inhibitor acts during processing of the protein and persistence of the farnesylated form in the presence of inhibitor (FIG. 16A) as well as presence of the unfarnesylated form after withdrawal of the inhibitor, are both longer in these human cells than in REF cells. Unfarnesylated H-ras was detectable after 5 hours of treatment with 5 μM FTI-277, but unlike ras transformed REF, 24 hours was required to achieve 50% of ras in the unfarnesylated form in human cells.

In view of the fact that accumulation of unfarnesylated H-ras in T24 cells takes longer, these cells were pretreated for 24 hours prior to irradiation and subsequent assay for clonogenic survival. An additional consideration raised by pretreatment of cells is that FTI-277 and other farnesyltransferase inhibitors can affect the farnesylation of rho proteins involved in cell morphology and thus alter cell adherence. The pretreatment of T24 cells with FTI-277 reduced the ability of these cells to form discrete colonies after replating, resulting in the formation of secondary satellite colonies. To avoid this complication, survival assays on pretreated T24 cells were performed in 96 well microtiter dishes using multiple dilutions of cells. The results of this assay demonstrate a significant reduction in colony formation after FTI-277 treatment and irradiation with 2 Gray with a concomitant reduction in the surviving fraction at 2 Gray irradiation from 0.58 to 0.35 (FIG. 16B). FTI-277 treatment alone did not significantly reduce the fraction of wells giving rise to colonies. These results demonstrate that FTI-277 can radiosensitize human cells that naturally express an activated H-ras as a result of mutation, and further show that the radiosensitization of human cells expressing activated ras can be detected at radiation doses that are used in radiotherapy.

Inhibition in K-ras Prenylation after FTI-277 Treatment of Human SW480 Cells

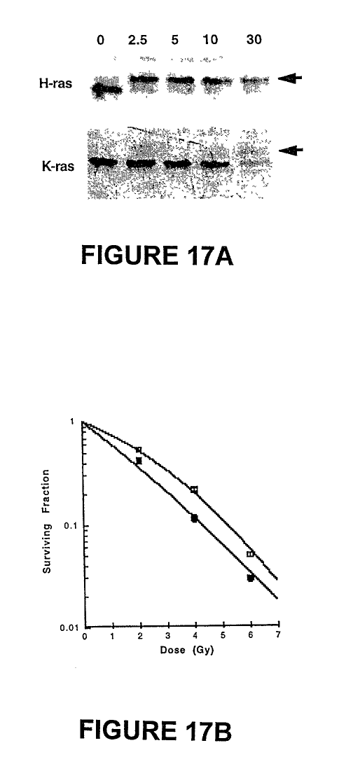

The effect of FTI 277 treatment is largely specific for H-ras rather than K-ras, inhibition of K-ras prenylation by FTI-277 was examined. As shown in FIG. 17A, the SW480 colon carcinoma cell line expressing H-ras and K-ras exhibited altered migration of H-ras when as little as 2.5 μM FTI-277 was used, while altered migration of K-ras became evident only at 30 μM FTI-277. At this latter dose, FTI-277 inhibits both farnesylation and geranylgeranylation (Lerner et al., 1995, J. Biol. Chem. 270:26770-26773). Thus, while FTI-277 specifically inhibits farnesylation of H-ras and K-ras remains prenylated at doses of FTI-277 below 30 μM, at 30 μM of this inhibitor, some inhibition of K-ras prenylation was seen. SW480 cells were treated with a dose of 30 μM FTI-277 to determine whether these cells could be radiosensitized (FIG. 17B). The results of clonogenic survival assays demonstrated the possibility of radiosensitizing human tumor cells expressing activated K-ras using FTI-277 alone.

Inhibition of K-ras Prenylation in Human SW480 Colon Carcinoma Cells and Panc-I Pancreatic Cells by Combined Treatment with FTI-227 and GGTI-298

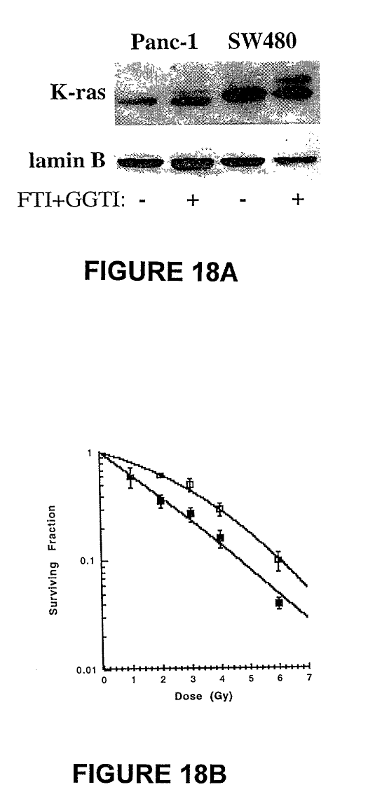

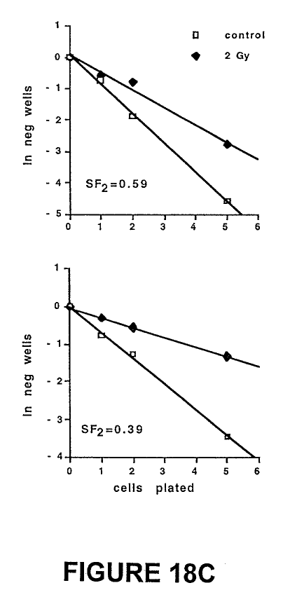

In view of the relatively high dose of FTI-277 required to inhibit K-ras prenylation, i.e., that dose where inhibition of GGTase I would also be expected, the combined use of an inhibitor of FTase and GGTase I was investigated to determine whether such a combination may be even more effective in conferring radiation sensitivity on cells. The results of these experiments establish that treatment of cells with the combination served to increase inhibition of K-ras prenylation (FIG. 18A). The concentration of FTI-277 required to inhibit K-ras prenylation was six-fold lower when combined with 8 μM GGTI-298 than the concentration required for the equivalent inhibition of K-ras prenylation when FTI-277 was used alone (FIG. 17A). Treatment of cells with 8 μM GGTI-298 alone had no effect on prenylation of K-ras. Thus combined prenyltransferase inhibitor treatment had a synergistic effect on inhibiting K-ras prenylation. At the concentrations used, the combined inhibitor treatment appears to have specificity for activated K-ras prenylation, since another farnesylated protein, nuclear lamin B, was not affected. Using these concentrations of inhibitors in a clonogenic survival assay, the results establish that combined FTase and GGTase I inhibitor treatment of human cells was also effective in radiosensitizing the SW480 cell line (FIG. 18B). This observation was extended to other human tumors expressing activating mutations in K-ras. As shown in FIG. 18C, the A549 lung carcinoma cell line also exhibited significant radiosensitization at 2 Gray following combined treatment with FTase and GGTase I inhibitors.

Thus, an FTase inhibitor such as FTI-277, is effective in radiosensitizing K-ras expressing human tumor cells. In addition, combined treatment of cells with an FTase and an GGTase I inhibitor served to inhibit prenylation of activated K-ras and to further enhance the radiosensitivity of human cells expressing a K-ras oncogene product. The radiosensitization effect of this treatment has been shown to be effective in both colon and lung carcinoma cells expressing activated ras oncogenes.

Although the degree of radiosensitization obtained after prenyltransferase treatment was not large, fractionated doses such as those used in clinical radiotherapy, can cause even small increases in cell killing which will appreciably improve the clinical outcome of the patient. This is the case because clinical radiotherapy involves the delivery of small daily doses of radiation over many weeks of treatment. The treatment proposed herein has the effect of amplifying small differences in radiosensitivity to the power of the number of treatments delivered (typically 30 or more treatments are delivered when treatment is delivered with curative intent (Fertil et al., 1981, Int. J. Rad. Onc. Biol. Phys. 7:621-629; Steel et al., 1991, Radiotherapy and Oncology 20:71-83; Thames et al., 1992, Int. J. Rad. Onc. 22:241-246). Thus, even small differences in radiosensitivity may have a very large impact on clinical outcome in cancer treatment.

It is important to note that this has in fact been demonstrated to be true in a prospective clinical trial. In the study of cervical cancer by West et al., a difference in median SF₂ between 0.38 and 0.54 was significant for survival at the p=0.01 level (West et al., 1995, Int. J. Rad. Onc. 31:841-846). This difference is less than the difference in SF₂ which has been observed in the experiments described herein using FTI-277 in human cells expressing activated ras. For this reason, it is believed that the degree of radiosensitization obtained in the experiments described herein may translate into a significant increase in radiosensitivity in a clinical setting. Examination of the effects of prenyltransferase inhibitors in fractionated irradiation schedules in a clinical setting will provide precise procedures and dosages for the administration of prenylation inhibitors in combination with irradiation for cancer therapy.

Inhibition of H-ras Prenylation in vivo using Human Colon Carcinomas Grown in Nude Mice

To examine the effectiveness of prenylation inhibitors in vivo in an animals, both rodent and human tumor responses to these inhibitors were examined in tumor grafts growing on nude mice. The protocols described in Hoffinan (1992, Curr, Perspect. on Molec. & Cell. Oncol. 1:311-326) were followed to establish and grow tumors in nude mice.

To perform the assay, tumors were implanted and allowed to grow to approximately 125 mm³. Mice having these tumors were then administered 50 mg/kg FTI-277 by intraperitoneal injection. Controls included mice which were injected with an equal volume of drug-free carrier solution. Two injections were given to each mouse, one at 18 hours and a second at 5 hours prior to sacrificing each mouse. The tumor ras prenylation status of the mice was subsequently assessed as described herein.

The feasibility of analyzing ras protein expression in tumor samples was first established by examining expression of H-ras from samples obtained from the 5R cell line which was grown as a tumor in nude mice. As shown in FIG. 19A, the expression of H-ras was easily detected in cell lysates obtained directly from tumor samples. Detected H-ras was derived from the tumor as assessed in Western blot analysis of normal mouse liver (FIG. 19A), skin or spleen, wherein the same monoclonal antibody used to detect H-ras in the tumor cells failed to detect H-ras expression in these tissues. H-ras may also be detected in human colon carcinoma tumors grown in nude mice. Further, the unfarnesylated form of H-ras was detectable following treatment of the animals in vivo with an FTase inhibitor (FIG. 19B).

The assays just described establish that ras protein expression may be detected in tumors in vivo in an animal and further, that alterations in ras mobility following FTase treatment in tumors of rodent and human origin grown in nude mice may also be detected. These assays therefore demonstrate the effectiveness of prenyltransferase inhibitors in vivo in an animal.

The disclosures of each and every patent, patent application and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of conferring radiation sensitivity on a tumor cell comprising administering to said cell at least one inhibitor of a protein product which participates in the ras signalling pathway, whereby inhibition of said protein product confers radiation sensitivity on said cell.

2. The method of claim 1, wherein said protein product is an oncogene protein product.

3. The method of claim 2, wherein said oncogene protein product is a ras protein.

4. The method of claim 3, wherein said ras protein is selected from the group consisting of H-ras, K_{A}-ras, K_{B}-ras and N-ras.

5. The method of claim 1, wherein said protein product is selected from the group consisting of rhoA, rhoB, rhoC and RAC-1.

6. The method of claim 1, wherein said inhibitor is an antisense oligonucleotide.

7. The method of claim 1, wherein said inhibitor is a ribozyme.

8. The method of claim 1, wherein said protein product has at the carboxyl terminus of the protein the sequence CAAX, wherein C is cysteine, A is an aliphatic amino acid, valine, leucine or isoleucine and X is methionine, serine, cysteine, alanine, glutamine, leucine or isoleucine.

9. The method of claim 8, wherein said inhibitor is a protein prenylation inhibitor.

10. The method of claim 9, wherein said prenylation inhibitor is a farnesylation inhibitor.

11. The method of claim 10, wherein said farnesylation inhibitor is selected from the group consisting of FTI-276 and FTI-277.

12. The method of claim 11, wherein the sulfate groups on FTI-276 and FTI-277 have been removed.

13. The method of claim 9, wherein said prenylation inhibitor is a geranylgeranylation inhibitor.

14. The method of claim 13, wherein said geranylgeranylation inhibitor is selected from the group consisting of GGTI-297 and GGTI-298.

15. The method of claim 14, wherein the sulfate groups on GGTI-297 and GGTI-298 have been removed.

16. The method of claim 1, wherein said tumor cell is a tumor cell in a solid tumor.

17. The method of claim 16, wherein said solid tumor is selected from the group consisting of prostate, lung, colon, breast, pancreas, cervical carcinoma, cervical sarcoma, rectum, colon, ovary, bladder, thyroid, head and neck.

18. The method of claim 17, wherein said solid tumor is selected from the group consisting of lung, pancreas, colon and rectum.

19. A method of reducing the growth of a tumor in an animal comprising
administering to said animal at least one inhibitor of a protein product expressed in cells of said tumor, which protein product participates in the ras signalling pathway and whereby inhibition of said protein product confers radiation sensitivity on said cells,
wherein said inhibitor is administered to said animal in an amount sufficient to effect inhibition of said protein product, and
irradiating said animal thereby reducing the growth of said tumor in said animal.

20. The method of claim 19, wherein said animal is a human.

21. The method of claim 19, wherein said protein product is an oncogene protein product.

22. The method of claim 21, wherein said oncogene protein product is a ras protein.

23. The method of claim 22, wherein said ras protein is selected from the group consisting of H-ras, K_{A}-ras, K_{B}-ras and N-ras.

24. The method of claim 19, wherein said protein product is selected from the group consisting of rhoA, rhoB, rhoC and RAC-1.

25. The method of claim 19, wherein said inhibitor is an antisense oligonucleotide.

26. The method of claim 19, wherein said inhibitor is a ribozyme.

27. The method of claim 19, wherein said protein product has at the carboxyl terminus of the protein the sequence CAAX, wherein C is cysteine, A is an aliphatic amino acid, valine, leucine or isoleucine and X is methionine, serine, cysteine, alanine, glutamine, leucine or isoleucine.

28. The method of claim 27, wherein said inhibitor is a protein prenylation inhibitor.

29. The method of claim 28, wherein said prenylation inhibitor is a farnesylation inhibitor.

30. The method of claim 29, wherein said farnesylation inhibitor is selected from the group consisting of FTI-276, FTI-277.

31. The method of claim 30, wherein the sulfate groups on FTI-276 and FTI-277 have been removed.

32. The method of claim 28, wherein said prenylation inhibitor is a geranylgeranylation inhibitor.

33. The method of claim 32, wherein said geranylgeranylation inhibitor is selected from the group consisting of GGTI-297 and GGTI-298.

34. The method of claim 33, wherein the sulfate groups on GGTI-297 and GGTI-298 have been removed.

35. The method of claim 19, wherein said tumor is a solid tumor.

36. The method of claim 35, wherein said solid tumor is selected from the group consisting of prostate, lung, colon, breast, pancreas, cervical carcinoma, cervical sarcoma, rectum, colon, ovary, bladder, thyroid, head and neck.

37. The method of claim 36, wherein said solid tumor is selected from the group consisting of lung, pancreas, colon and rectum.

38. A method of eliminating a tumor from an animal comprising
administering to said animal at least one inhibitor of a protein product expressed in cells of said tumor, which protein product participates in the ras signalling pathway and whereby inhibition of said protein product confers radiation sensitivity on said cells,
wherein said inhibitor is administered to said animal in an amount sufficient to effect inhibition of said protein product, and
irradiating said animal thereby eliminating said tumor from said animal.

39. The method of claim 38, wherein said animal is a human.

40. The method of claim 38, wherein said protein product is an oncogene protein product.

41. The method of claim 40, wherein said oncogene protein product is a ras protein.

42. The method of claim 41, wherein said ras protein is selected from. the group consisting of H-ras, K_{A}-ras, K_{B}-ras and N-ras.

43. The method of claim 38, wherein said protein product is selected from the group consisting of rhoA, rhoB, rhoC and RAC-1.

44. The method of claim 38, wherein said inhibitor is an antisense oligonucleotide.

45. The method of claim 38, wherein said inhibitor is a ribozyme.

46. The method of claim 38, wherein said protein product has at the carboxyl terminus of the protein the sequence CAAX, wherein C is cysteine, A is an aliphatic amino acid, valine, leucine or isoleucine and X is methionine, serine, cysteine, alanine, glutamine, leucine or isoleucine.

47. The method of claim 46, wherein said inhibitor is a protein prenylation inhibitor.

48. The method of claim 47, wherein said prenylation inhibitor is a farnesylation inhibitor.

49. The method of claim 48, wherein said farnesylation inhibitor is selected from the group consisting of FTI-276 and FTI-277.

50. The method of claim 49, wherein the sulfate groups on FTI-276 and FTI-277 have been removed.

51. The method of claim 47, wherein said prenylation inhibitor is a geranylgeranylation inhibitor.

52. The method of claim 51, wherein said geranylgeranylation inhibitor is selected from the group consisting of GGTI-297 and GGTI-298.

53. The method of claim 52, wherein the sulfate groups on GGTI-297 and GGTI-298 have been removed.

54. The method of claim 38, wherein said tumor is a solid tumor.

55. The method of claim 54, wherein said solid tumor is selected from the group consisting of prostate, lung, colon, breast, pancreas, cervical carcinoma, cervical sarcoma, rectum, colon, ovary, bladder, thyroid, head and neck.

56. The method of claim 55, wherein said solid tumor is selected from the group consisting of lung, pancreas, colon and rectum.

57. A method of identifying a prenylation inhibitor which confers radiation sensitivity on a cell population comprising
providing a population of cells which express a protein in need of prenylation for activity of said protein and which protein participates in the ras signalling pathway,
adding to said cells a test compound,
irradiating said cells, and
measuring the level of sensitivity of said cells to irradiation, wherein a higher level of radiation sensitivity in cells administered the test compound compared with the level of radiation sensitivity in cells which were not administered the test compound, is an indication that said test compound confers radiation sensitivity on said cell population.

58. The method of claim 57, wherein said protein has at the carboxyl terminus of the protein the sequence CAAX, wherein C is cysteine, A is an aliphatic amino acid, valine, leucine or isoleucine and X is methionine, serine, cysteine, alanine, glutamine, leucine or isoleucine.

59. The method of claim 58, wherein said protein is selected from the group consisting of rhoA, rhoB, rhoC and RAC-1.

60. The method of claim 58, wherein said protein is an oncogene protein product.

61. The method of claim 60, wherein said protein is a ras protein.

62. The method of claim 61, wherein said ras protein is selected from the group consisting of H-ras, K_{A}-ras, K_{B}-ras and N-ras.
